SEQUENCE LISTING (4 sequences)

SEQ ID NO: 1 - protein, 39 aa, Artificial Sequence
  Compound
  MISC_FEATURE (2)..(2): X is Aib in the description
  MISC_FEATURE (13)..(13): X is Aib in the description
  MISC_FEATURE (17)..(17): K is K1 in the description, K1 indicates that the amino group on the side chain of the lysine is connected to -X-X1-X2
  MISC_FEATURE (19)..(19): X is Z0 in the description, Z0 is selected from glutamine (Q) and asparagine (N)
  MISC_FEATURE (20)..(20): K is K0 in the description, K0 indicates that the carboxyl group on the side chain of the glutamic acid (E0) and the amino group on the side chain of the lysine (K0) jointly form a lactam structure
  MISC_FEATURE (21)..(21): X is Z1 in the descriptionZ1 is selected from alanine (A) and glutamic acid (E)
  MISC_FEATURE (23)..(23): X is Z2 in the description, Z2 is selected from valine (V) and isoleucine (I)
  MISC_FEATURE (24)..(24): E is E0 in the description, E0 indicates that the carboxyl group on the side chain of the glutamic acid (E0) and the amino group on the side chain of the lysine (K0) jointly form a lactam structure
  MISC_FEATURE (27)..(27): X is Z3 in the description, Z3 is selected from isoleucine (I) and leucine (L)
  MISC_FEATURE (39)..(39): S is S0 in the description
YXEGTFTSDYSIXLDKKAXKXFXEWLXAGGPSSGAPPPS

SEQ ID NO: 2 - protein, 39 aa, Artificial Sequence
  Compound
  MISC_FEATURE (2)..(2): X is Aib in the description
  MISC_FEATURE (13)..(13): X is Aib in the description
  MISC_FEATURE (17)..(17): E is E0 in the description, E0 indicates that the carboxyl group on the side chain of the glutamic acid (E0) and the amino group on the side chain of the lysine (K0) jointly form a lactam structure
  MISC_FEATURE (20)..(20): K is K0 in the description, K0 indicates that the carboxyl group on the side chain of the glutamic acid (E0) and the amino group on the side chain of the lysine (K0) jointly form a lactam structure
  MISC_FEATURE (24)..(24): K is K1 in the description, K1 indicates that the amino group on the side chain of the lysine is connected to -X-X1-X2
  MISC_FEATURE (39)..(39): S is S0 in the description
YXEGTFTSDYSIXLDKEAQKAFVKWLIAGGPSSGAPPPS

SEQ ID NO: 3 - protein, 39 aa, Artificial Sequence
  Compound
  MISC_FEATURE (2)..(2): X is Aib in the description
  MISC_FEATURE (13)..(13): E is E0 in the description, E0 indicates that the carboxyl group on the side chain of the glutamic acid (E0) and the amino group on the side chain of the lysine (K0) jointly form a lactam structure
  MISC_FEATURE (17)..(17): K is K0 in the description, K0 indicates that the carboxyl group on the side chain of the glutamic acid (E0) and the amino group on the side chain of the lysine (K0) jointly form a lactam structure
  MISC_FEATURE (20)..(20): K is K1 in the description, K1 indicates that the amino group on the side chain of the lysine is connected to -X-X1-X2
  MISC_FEATURE (39)..(39): S is S0 in the description
YXEGTFTSDYSIELDKKAQKAFVQWLIAGGPSSGAPPPS

SEQ ID NO: 4 - protein, 39 aa, Artificial Sequence
  Compound
  MISC_FEATURE (2)..(2): X is Aib in the description
  MISC_FEATURE (13)..(13): E is E0 in the description, E0 indicates that the carboxyl group on the side chain of the glutamic acid (E0) and the amino group on the side chain of the lysine (K0) jointly form a lactam structure
  MISC_FEATURE (16)..(16): K is K0 in the description, K0 indicates that the carboxyl group on the side chain of the glutamic acid (E0) and the amino group on the side chain of the lysine (K0) jointly form a lactam structure
  MISC_FEATURE (20)..(20): K is K1 in the description, K1 indicates that the amino group on the side chain of the lysine is connected to -X-X1-X2
  MISC_FEATURE (39)..(39): S is S0 in the description
YXEGTFTSDYSIELDKIAQKAFVQWLIAGGPSSGAPPPS

(19) United States
(12) Patent Application Publication
(10) Pub. No.: US 2024/0067702 A1
(43) Pub. Date: Feb. 29, 2024

(54) LACTAM-MODIFIED POLYPEPTIDE COMPOUNDS

(71) Applicant: DONGBAO PURPLE STAR (HANGZHOU) BIOPHARMACEUTICAL CO., LTD., Hangzhou, Zhejiang (CN)

(72) Inventors: Zhixiang PAN, Shanghai (CN); Zhigan JIANG, Shanghai (CN); Haiying HE, Shanghai (CN); Guoping HU, Shanghai (CN); Jian LI, Shanghai (CN); Shuhui CHEN, Shanghai (CN)

(21) Appl. No.: 18/039,582

(22) PCT Filed: Dec. 2, 2021
(86) PCT No.: PCT/CN2021/135180

§ 371 Date: May 31, 2023

(30) Foreign Application Priority Data
Dec. 2, 2020 (CN) 202011402979.7
Dec. 2, 2020 (CN) 202011409947.X
Apr. 21, 2021 (CN) 202110432060.0
May 27, 2021 (CN) 202110587056.1

(51) Int. Cl. C07K 14/72; A61P 3/04; A61P 3/10

(52) U.S. Cl. CPC C07K 14/72; A61P 3/04; A61P 3/10; A61K 38/00

(57) ABSTRACT

A class of lactam-modified polypeptide compounds and the use thereof in the preparation of a drug for treating related diseases.

The present application claims the right of the following priorities:
CN202011402979.7, the application date is Dec. 2, 2020;
CN202011409947.X, the application date is Dec. 2, 2020;
CN202110432060.0, the application date is Apr. 21, 2021;
CN202110587056.1, the application date is May 27, 2021.

### TECHNICAL FIELD

The present disclosure relates to a class of polypeptide compounds containing lactam modification, and the use thereof in the manufacture of a medicament for treating related diseases.

### BACKGROUND

It is estimated that there are 110 million diabetics in China, accounting for 24% of diabetics in the world. With economic development and lifestyle changes, the prevalence of diabetes in China has increased to 12.8% (as high as 19.9% in some provinces and cities), which is accompanied by obesity or cardiovascular disease (CVD). Recent studies have found that a glucose-dependent insulinotropic peptide (GIP)/glucagon-like peptide-1 (GLP-1) dual agonist can be used for treating diabetes.

GLP-1 plays a role in protecting islet β cells in islets and effectively controls postprandial blood glucose by stimulating the islet β cells to release insulin in a glucose-dependent manner. Due to GLP-1's unique mechanism of action, the risk of hypoglycemia is greatly reduced. Although GLP-1R agonists have demonstrated excellent glucose-lowering effect in clinical practice, there are still many type 2 diabetics who have not achieved their glycemic and weight goals. Therefore, combining GLP-1R with other glucose-lowering targets such as GIP for treating type 2 diabetes is an urgent and promising need. GIP is a polypeptide secreted by neuroendocrine K cells of small intestine, and GIP's physiological actions are mediated by GIPR including non-glucose-dependent insulinotropic effects, stimulation of glucagon secretion, and enhancement of lipid metabolism, etc. Although the beneficial effects of GIPR agonists appear to be attenuated in hyperglycemic symptoms in type 2 diabetics, studies have shown that the diminished insulinotropic effect of GIP can be fully restored after a period of normalization of plasma glucose levels. This indicates that co-agonism of GLP-1R/GIPR can exert a synergistic glucose-lowering effect. A GIP and GLP-1 dual agonist has a synergistic effect on the regulation of glucose/lipid metabolism and has better therapeutic effects on lowering blood glucose, reducing body weight, and alleviating liver fat.

### CONTENT OF THE PRESENT INVENTION

The present disclosure provides a compound represented by the following formula or a pharmaceutically acceptable salt thereof,
SEQ ID NO1: YAibEGT FTSDY SIAibLD KK¹AZ₀K₀ Z₁FZ₂E₀W LZ₃AGG PSSGA PPPS₀
SEQ ID NO2: YAibEGT FTSDY SIAibLD KE₀AQK₀ AFVK¹W LIAGG PSSGA PPPS₀
SEQ ID NO3: YAibEGT FTSDY SIE₀LD KK₀AQK¹ AFVQW LIAGG PSSGA PPPS₀
SEQ ID NO4: YAibEGT FTSDY SIE₀LD K₀IAQK¹ AFVQW LIAGG PSSGA PPPS₀
wherein
Aib has a structure of

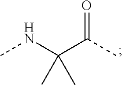

S₀ is selected from

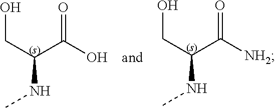

Z₀ is selected from glutamine (Q) and asparagine (N);
Z₁ is selected from alanine (A) and glutamic acid (E);
Z₂ is selected from valine (V) and isoleucine (I);
Z₃ is selected from isoleucine (I) and leucine (L);
E₀ and K₀ indicate that the carboxyl group on the side chain of the glutamic acid and the amino group on the side chain of the lysine jointly form a lactam structure, and in this case, K₀Z₁FZ₂E₀ has a structure of

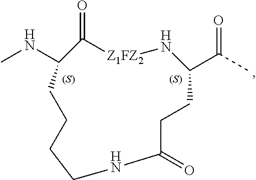

E₀AQK₀ has a structure of

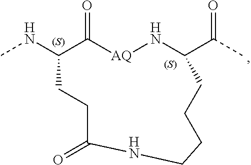

E₀LDKK₀ has a structure of

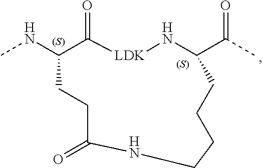

and E₀LDK₀ has a structure of

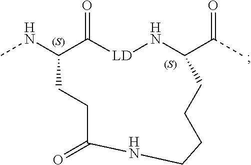

K¹ indicates that the amino group on the side chain of the lysine is connected to —X—X1-X2, and has a structure of

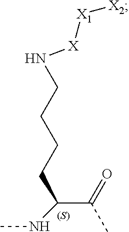

X is selected from

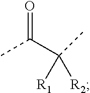

R₁ and R₂ are each independently selected from H and CH₃;
X₁ is selected from

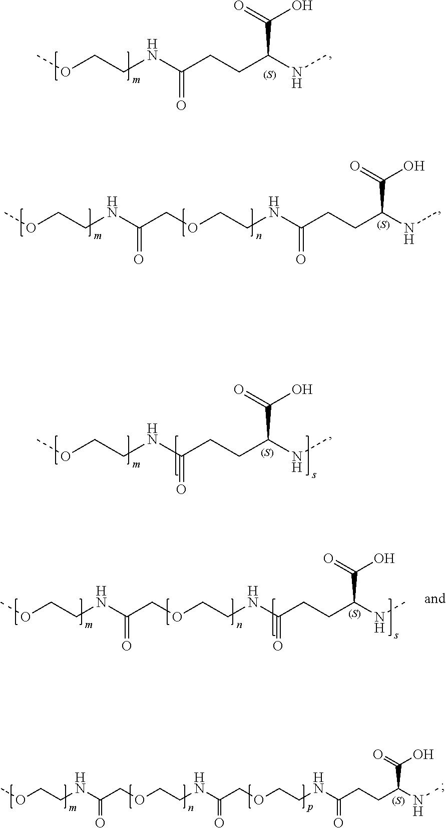

X₂ is selected from

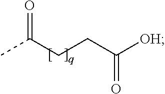

m, n and p are each independently selected from 2 and 3;
s is selected from 2 and 3;
q is selected from 15, 16, 17, 18 and 19.

In some embodiments of the present disclosure, the compound or the pharmaceutically acceptable salt thereof is selected from:

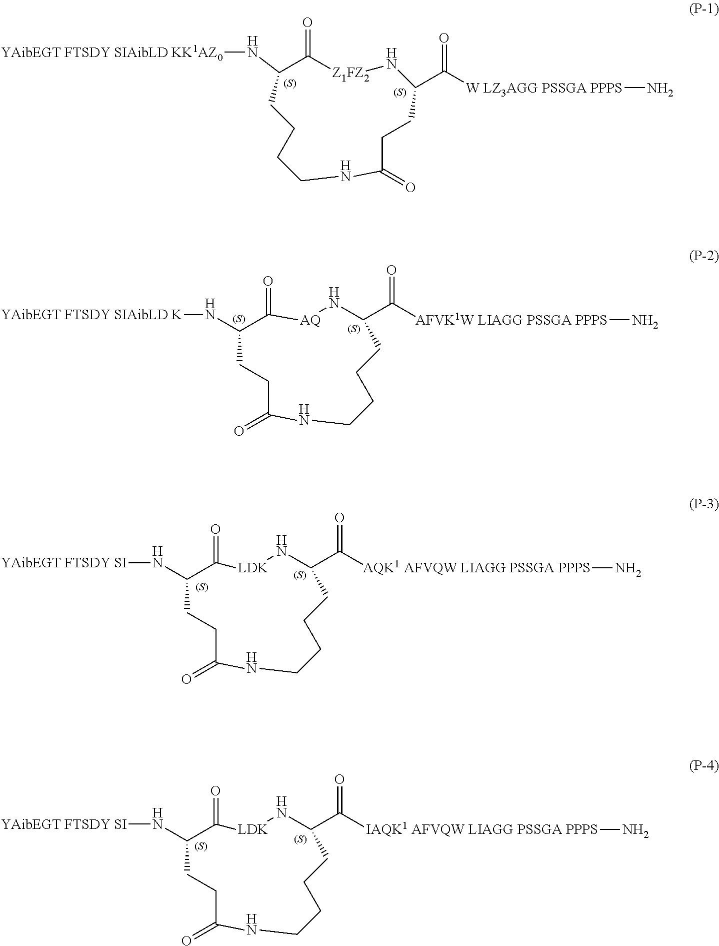

wherein
Aib, Z₀, Z₁, Z₂, Z₃ and K¹ are as defined in the present disclosure.

In some embodiments of the present disclosure, the X is selected from —C(═O)—CH₂—, and other variables are as defined in the present disclosure.

In some embodiments of the present disclosure, the m, n and p are each independently selected from 2, and other variables are as defined in the present disclosure.

In some embodiments of the present disclosure, the s is selected from 2, and other variables are as defined in the present disclosure.

In some embodiments of the present disclosure, the q is selected from 15 and 17, and other variables are as defined in the present disclosure.

In some embodiments of the present disclosure, the X₁ is selected from

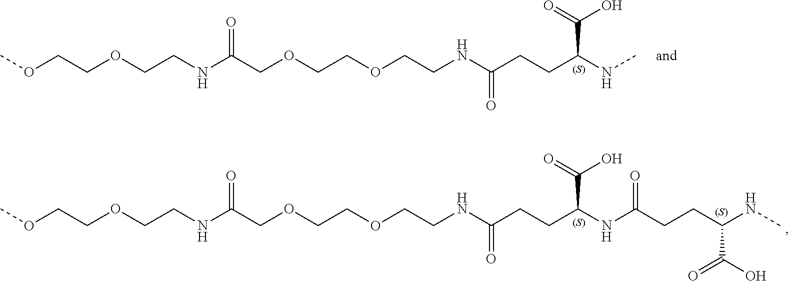

and other variables are as defined in the present disclosure.

In some embodiments of the present disclosure, the X₂ is selected from

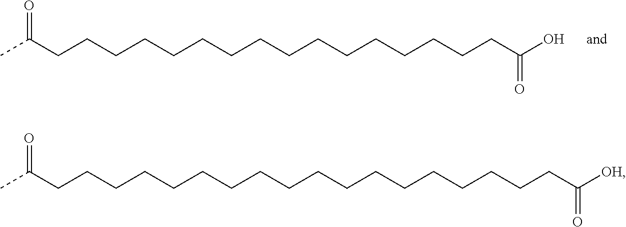

and other variables are as defined in the present disclosure.

In some embodiments of the present disclosure, the —X—X₁—X₂ is selected from

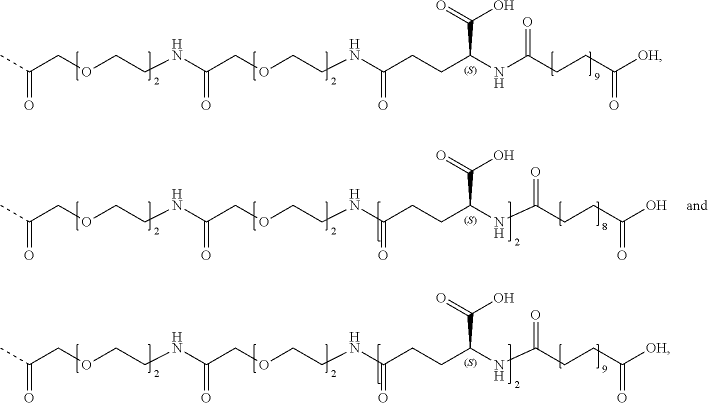

and other variables are as defined in the present disclosure.

The present disclosure provides a compound represented by the following formula or a pharmaceutically acceptable salt thereof,
YAibEGT FTSDY SIAibLD KK¹AQK₀ AFVE₀W LIAGG PSSGA PPPS₀
YAibEGT FTSDY SIAibLD KK¹ANK₀ AFVE₀W LIAGG PSSGA PPPS₀
YAibEGT FTSDY SIAibLD KK¹AQK₀ EFVE₀W LIAGG PSSGA PPPS₀
YAibEGT FTSDY SIAibLD KK¹AQK₀ AFIE₀W LIAGG PSSGA PPPS₀
YAibEGT FTSDY SIAibLD KK¹AQK₀ AFVE₀W LLAGG PSSGA PPPS₀
wherein
Aib has a structure of

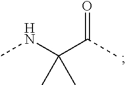

S₀ is selected from

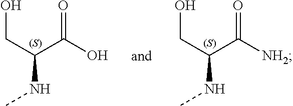

K₀ and E₀ indicate that the amino group on the side chain of the lysine and the carboxyl group on the side chain of the glutamic acid jointly form a lactam structure, and have a structure of

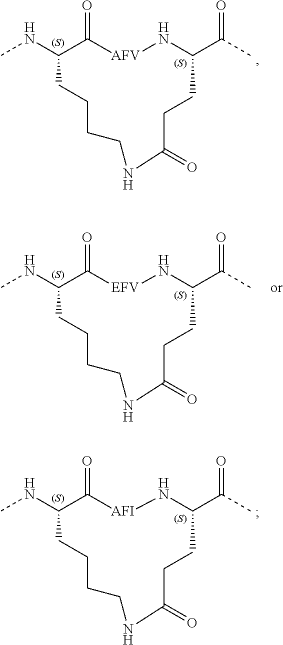

K¹ indicates that the amino group on the side chain of the lysine is connected to —X—X₁—X₂, and has a structure of

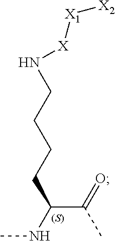

X is selected from

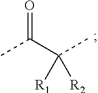

R₁ and R₂ are each independently selected from H;
X₁ is selected from

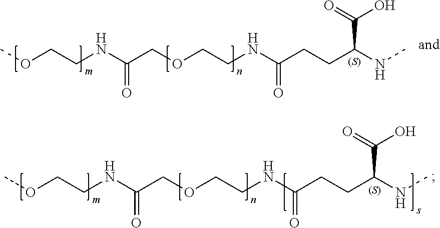

X₂ is selected from

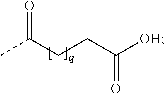

m and n are each independently selected from 2 and 3;
s is selected from 2 and 3;
q is selected from 15, 16, 17, 18 and 19.

In some embodiments of the present disclosure, the X is selected from —C(═O)—CH₂—, and other variables are as defined in the present disclosure.

In some embodiments of the present disclosure, the m and n are each independently selected from 2, and other variables are as defined in the present disclosure.

In some embodiments of the present disclosure, the s is selected from 2, and other variables are as defined in the present disclosure.

In some embodiments of the present disclosure, the q is selected from 15 and 17, and other variables are as defined in the present disclosure.

In some embodiments of the present disclosure, the X₁ is selected from

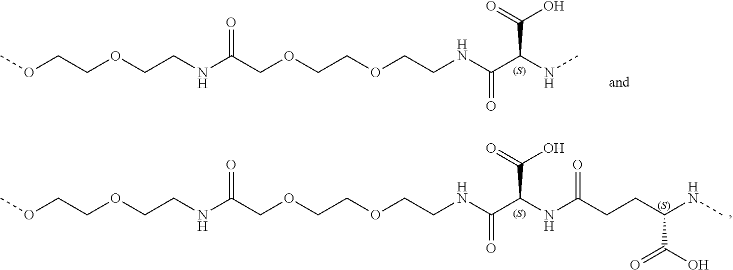

and other variables are as defined in the present disclosure.

In some embodiments of the present disclosure, the X₂ is selected from

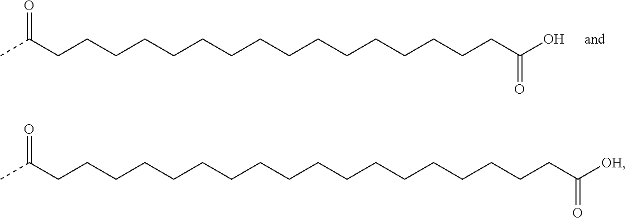

and other variables are as defined in the present disclosure.

In some embodiments of the present disclosure, the —X—X₁—X₂ is selected from

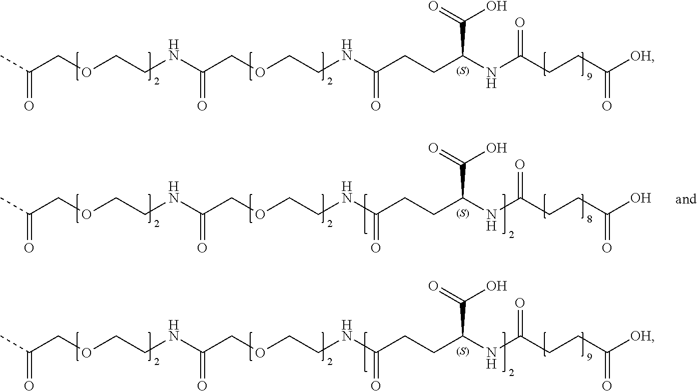

and other variables are as defined in the present disclosure.

The present disclosure provides a compound represented by the following formula or a pharmaceutically acceptable salt thereof,
YAibEGT FTSDY SIAibLD KK¹AZ₀K₀ Z₁FZ₂E₀W LZ₃AGG PSSGA PPPS₀
YAibEGT FTSDY SIAibLD KE₀AQK₀ AFVK¹W LIAGG PSSGA PPPS₀
YAibEGT FTSDY SIE₀LD KK₀AQK¹ AFVQW LIAGG PSSGA PPPS₀
wherein
Aib has a structure of

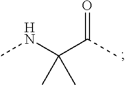

S₀ is selected from

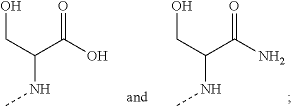

Z₀ is selected from glutamine (Q) and asparagine (N);
Z₁ is selected from alanine (A) and glutamic acid (E);
Z₂ is selected from valine (V) and isoleucine (I);
Z₃ is selected from isoleucine (I) and leucine (L);
E₀ and K₀ indicate that the carboxyl group on the side chain of the glutamic acid and the amino group on the side chain of the lysine jointly form a lactam structure, and in this case, K₀Z₁FZ₂E₀ has a structure of

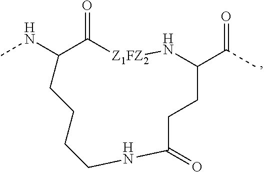

E₀AQK₀ has a structure of

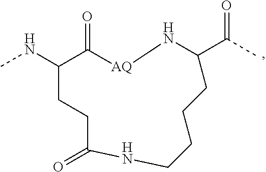

E₀LDKK₀ has a structure of

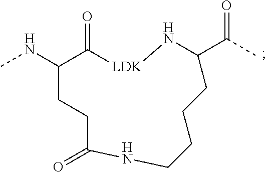

K¹ indicates that the amino group on the side chain of the lysine is connected to —X—X₁—X₂, and has a structure of

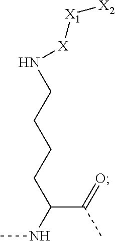

X is selected from

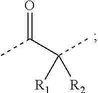

R₁ and R₂ are each independently selected from H;
X₁ is selected from

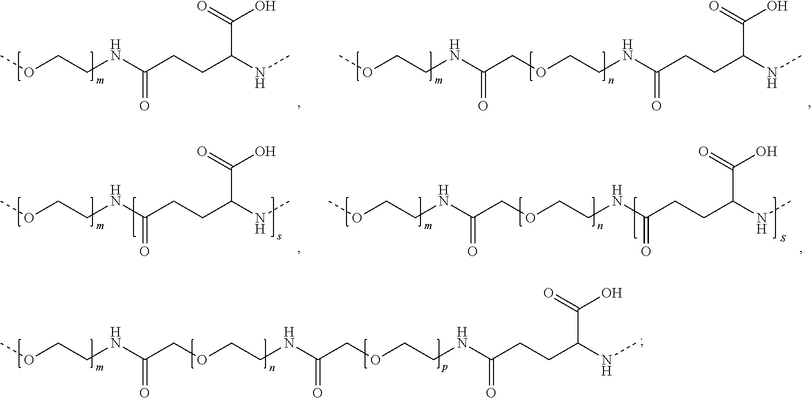

X₂ is selected from

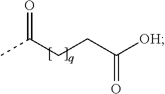

m, n and p are each independently selected from 2 and 3;
s is independently selected from 2 and 3;
q is independently selected from 15, 16, 17, 18 and 19.

In some embodiments of the present disclosure, the X is selected from —C(═O)—CH₂—, and other variables are as defined in the present disclosure.

In some embodiments of the present disclosure, the in, n and p are each independently selected from 2, and other variables are as defined in the present disclosure.

In some embodiments of the present disclosure, the s is independently selected from 2, and other variables are as defined in the present disclosure.

In some embodiments of the present disclosure, the q is independently selected from 15 and 17, and other variables are as defined in the present disclosure.

In some embodiments of the present disclosure, the q is independently selected from 17, and other variables are as defined in the present disclosure.

In some embodiments of the present disclosure, the X₁ is selected from

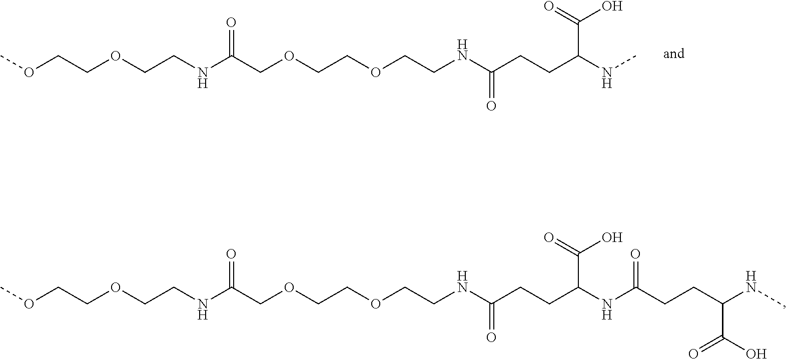

and other variables are as defined in the present disclosure.

In some embodiments of the present disclosure, the X₂ is selected from

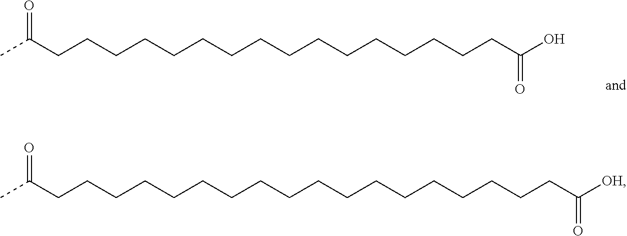

and other variables are as defined in the present disclosure.

In some embodiments of the present disclosure, the —X—X₁—X₂ is selected from

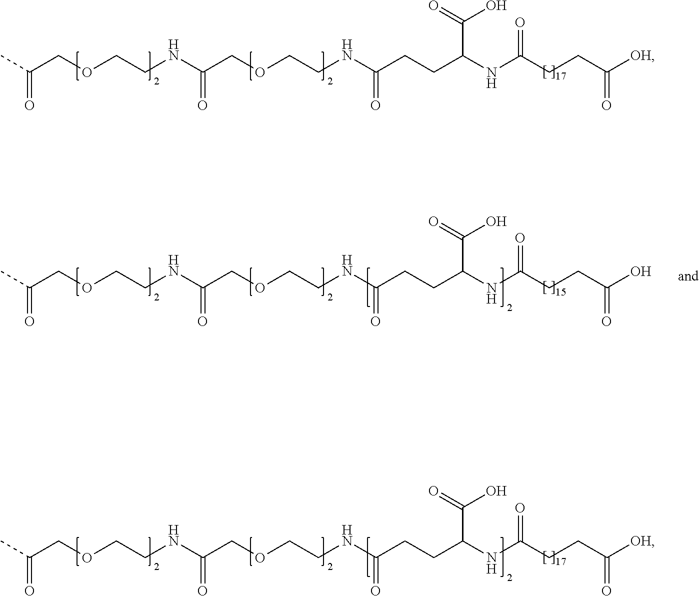

and other variables are as defined in the present disclosure.

The present disclosure provides a compound represented by the following formula or a pharmaceutically acceptable salt thereof,
YAibEGT FTSDY SIAibLD KK¹AQK₀ AFVE₀W LIAGG PSSGA PPPS₀
wherein
Aib has a structure of

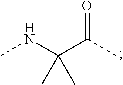

S₀ is selected from

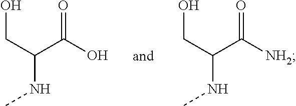

K₀ and E₀ indicate that the amino group on the side chain of the lysine and the carboxyl group on the side chain of the glutamic acid jointly form a lactam structure, and have a structure of

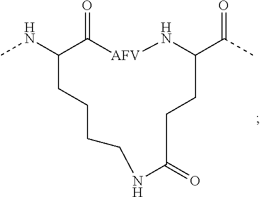

K¹ indicates that the amino group on the side chain of the lysine is connected to —X—X₁—X₂, and has a structure of

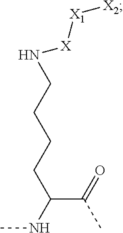

X is selected from

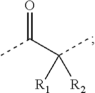

R₁ and R₂ are each independently selected from H;
X₁ is selected from

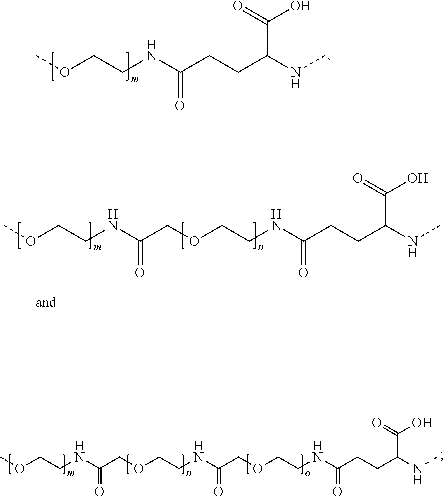

X₂ is selected from

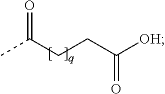

m, n and p are each independently selected from 2 and 3;
q is independently selected from 15, 16, 17, 18 and 19.

In some embodiments of the present disclosure, the X is selected from —C(═O)—CH₂—, and other variables are as defined in the present disclosure.

In some embodiments of the present disclosure, the m, n and p are each independently selected from 2, and other variables are as defined in the present disclosure.

In some embodiments of the present disclosure, the q is independently selected from 17, and other variables are as defined in the present disclosure.

In some embodiments of the present disclosure, the X₁ is selected from

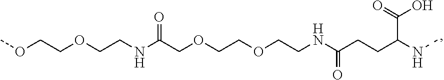

and other variables are as defined in the present disclosure.

In some embodiments of the present disclosure, the X₂ is selected from

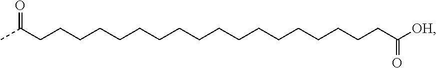

and other variables are as defined in the present disclosure.

In some embodiments of the present disclosure, the —X—X₁—X₂ is selected from

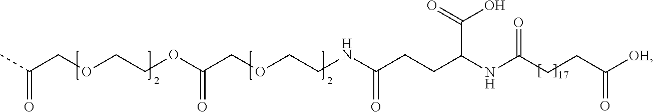

and other variables are as defined in the present disclosure.

The present disclosure provides a compound represented by the following formula or a pharmaceutically acceptable salt thereof,
YAibEGT FTSDY SIE₀LD K₀IAQK¹ AFVQW LIAGG PSSGA PPPS₀
wherein
Aib has a structure of

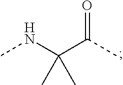

S₀ is selected from

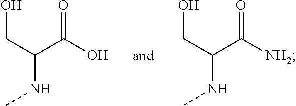

E₀ and K₀ indicate that the carboxyl group on the side chain of the glutamic acid and the amino group on the side chain of the lysine jointly form a lactam structure, and have a structure of

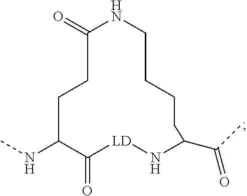

K¹ indicates that the amino group on the side chain of the lysine is connected to —X—X₁—X₂, and has a structure of

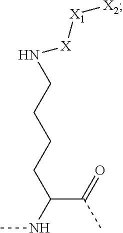

X is selected from

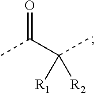

R₁ and R₂ are each independently selected from H;
X₁ is selected from

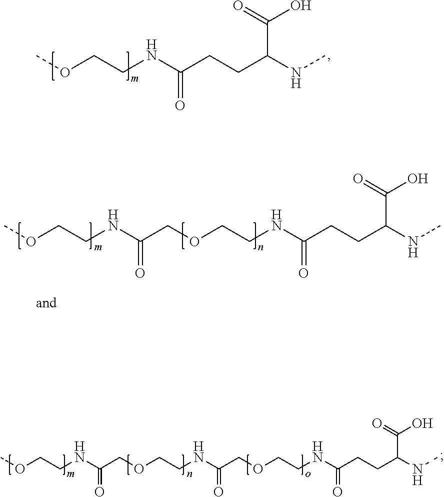

X₂ is selected from

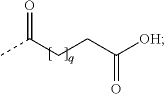

m, n and p are each independently selected from 2 and 3;
q is independently selected from 15, 16, 17, 18 and 19.

In some embodiments of the present disclosure, the X is selected from —C(═O)—CH₂—, and other variables are as defined in the present disclosure.

In some embodiments of the present disclosure, the m, n and p are each independently selected from 2, and other variables are as defined in the present disclosure.

In some embodiments of the present disclosure, the q is independently selected from 17, and other variables are as defined in the present disclosure.

In some embodiments of the present disclosure, the X₁ is selected from

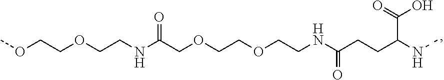

and other variables are as defined in the present disclosure.

In some embodiments of the present disclosure, the X₂ is selected from

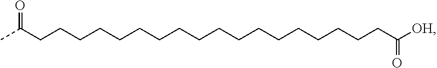

and other variables are as defined in the present disclosure.

In some embodiments of the present disclosure, the —X—X₁—X₂ is selected from

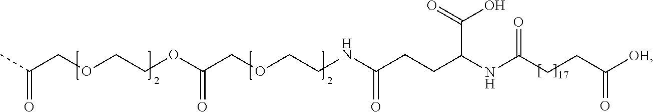

and other variables are as defined in the present disclosure.

There are still some embodiments of the present disclosure which are obtained by any combination of the above variables.

The present disclosure also provides a compound represented by the following formula or a pharmaceutically acceptable salt thereof,

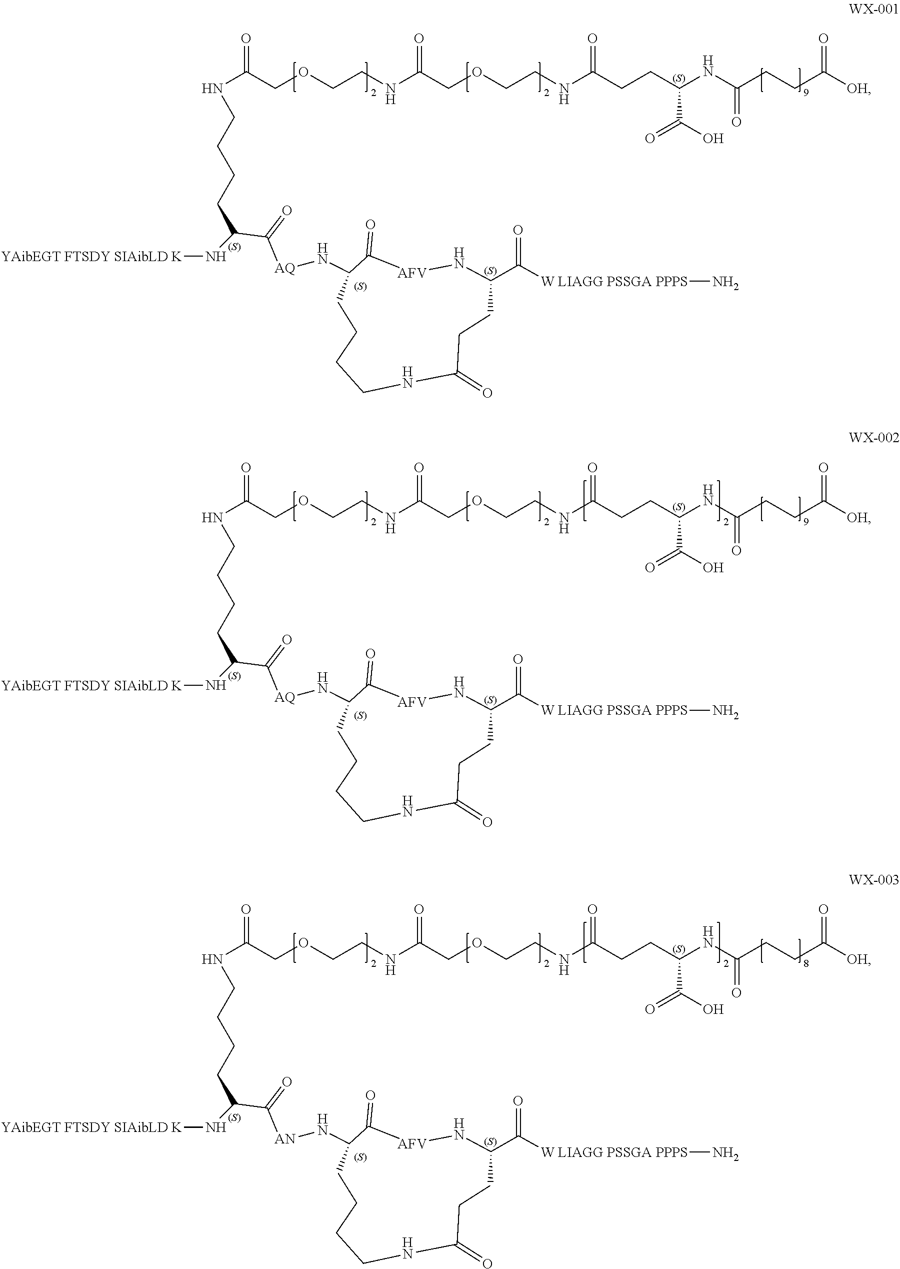

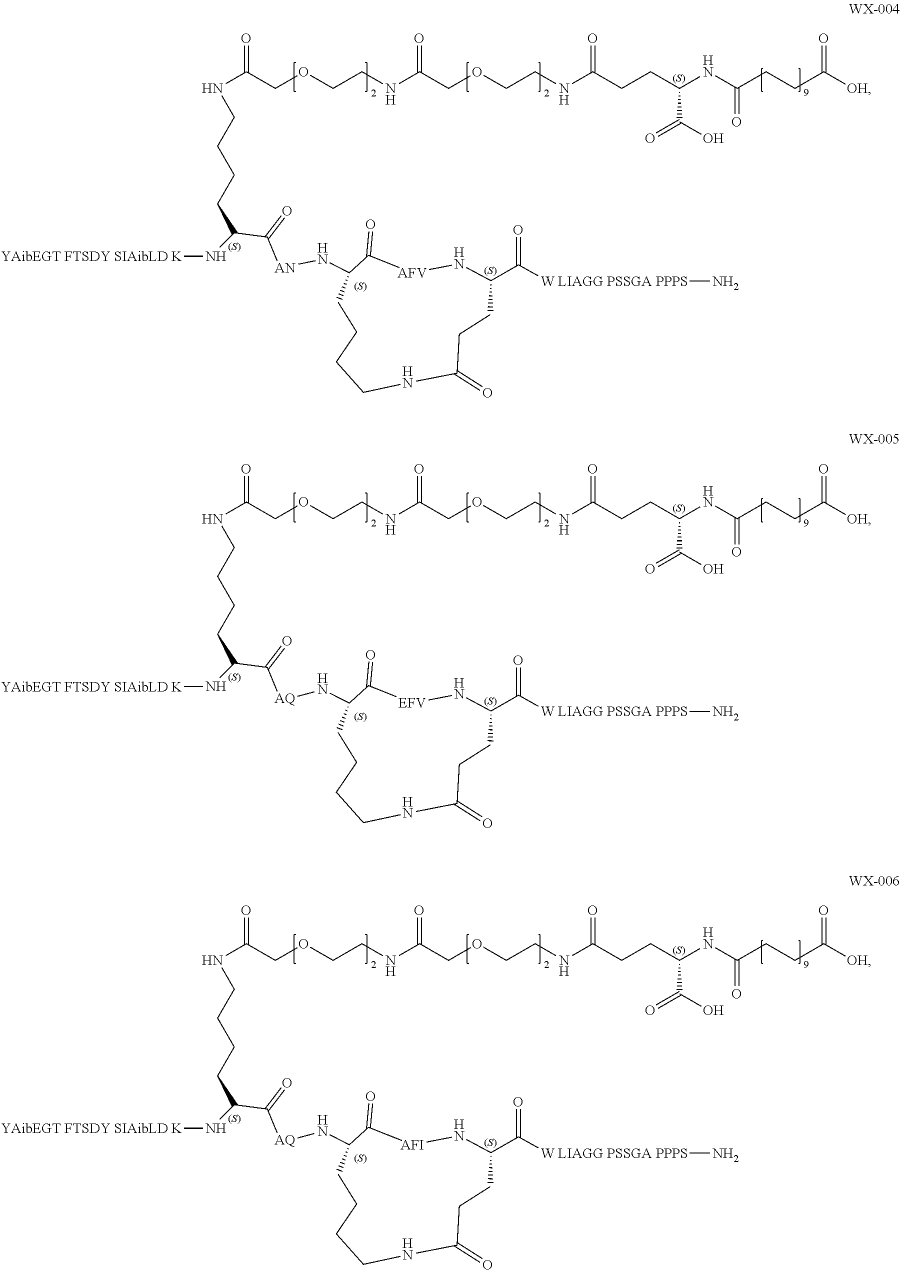

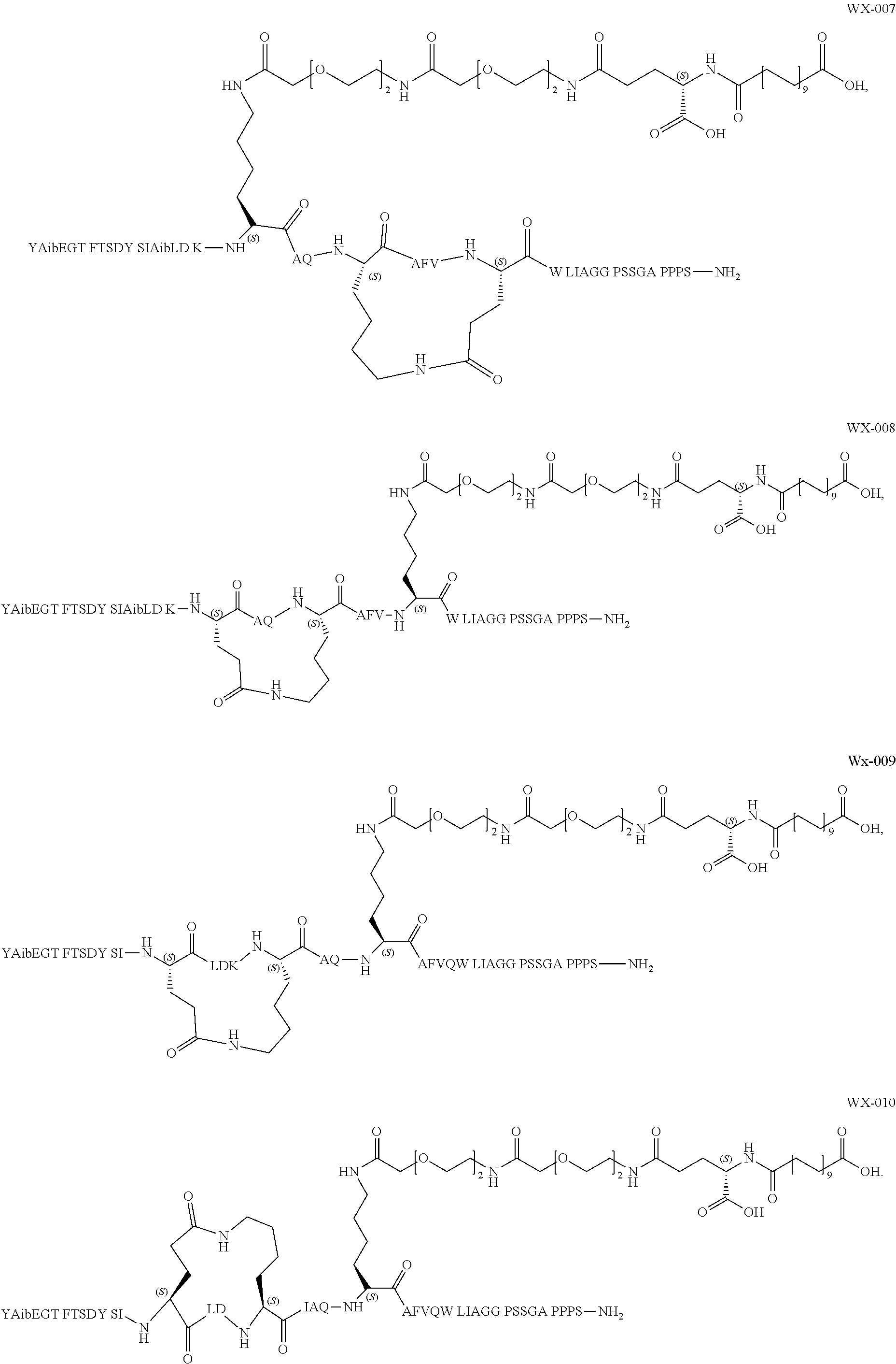

The present disclosure also provides a compound represented by the following formula or a pharmaceutically acceptable salt thereof,

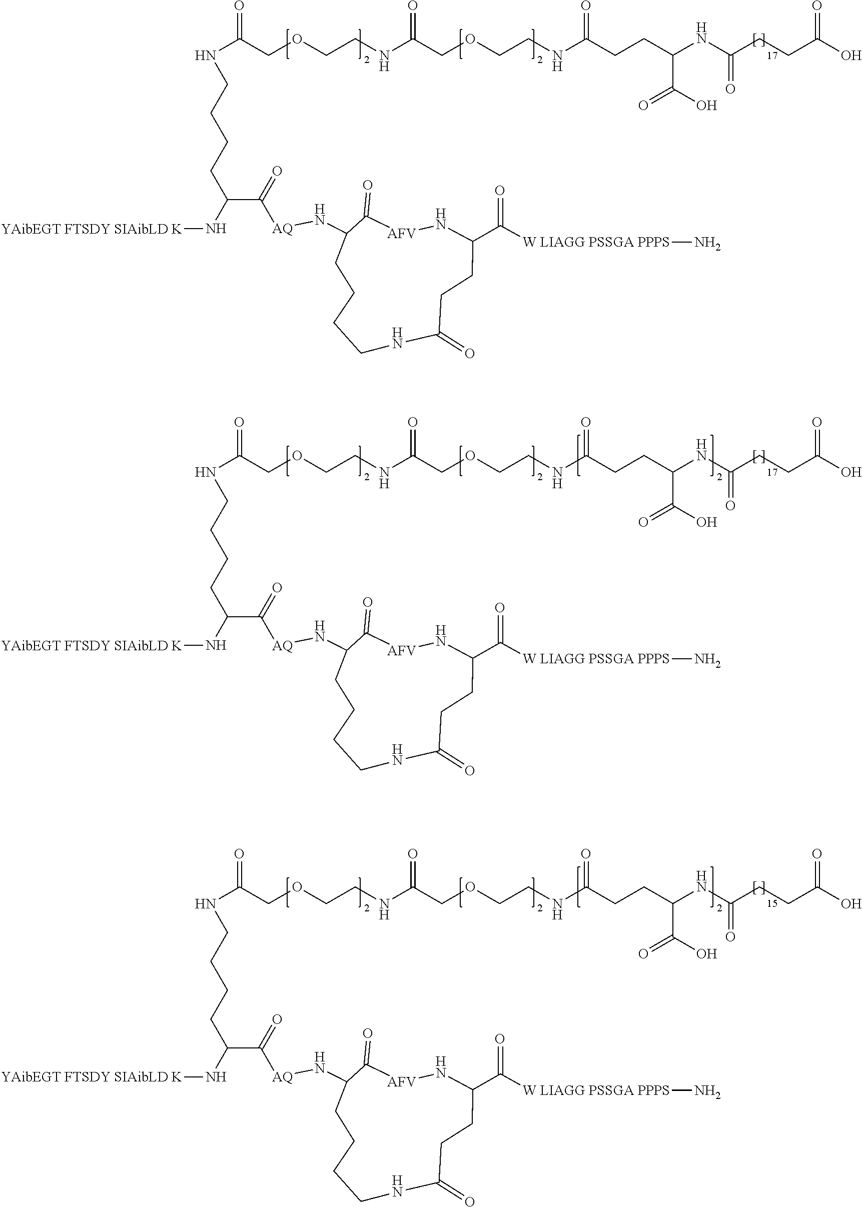

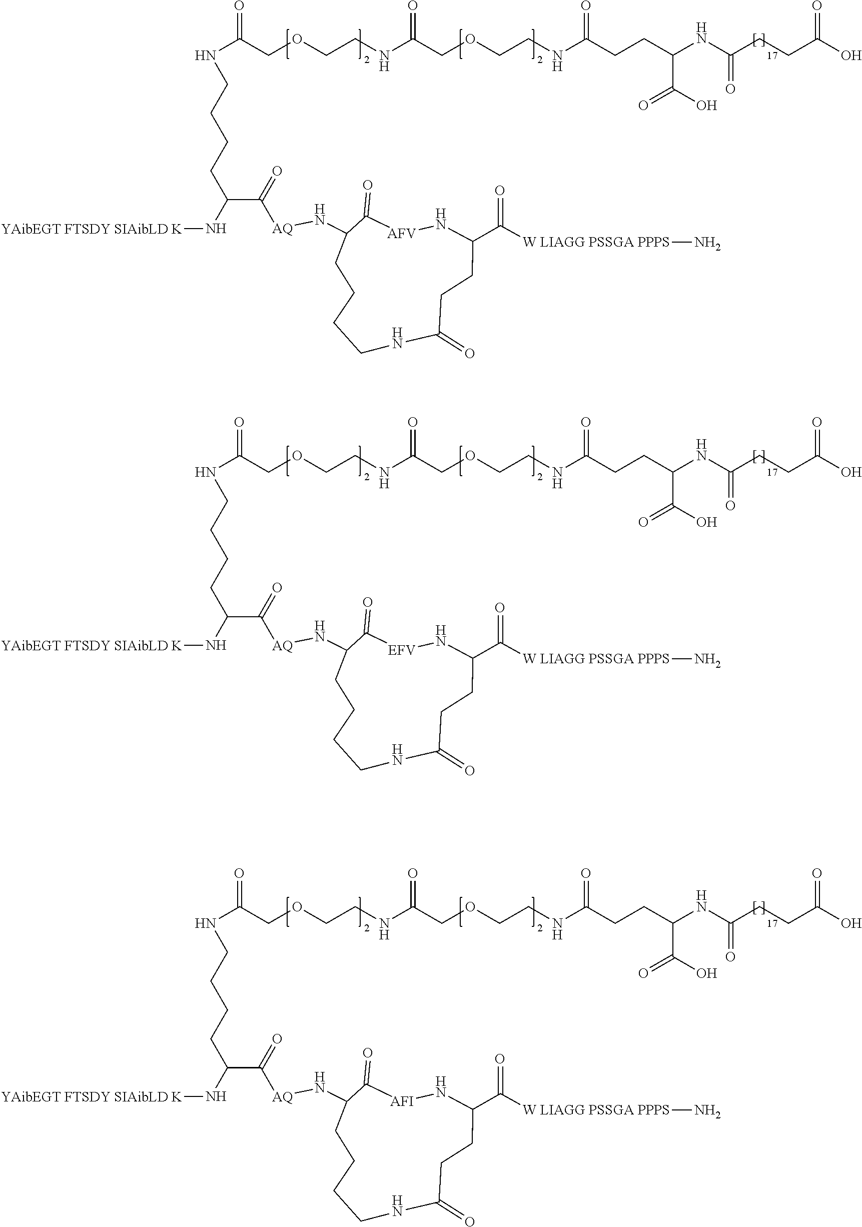

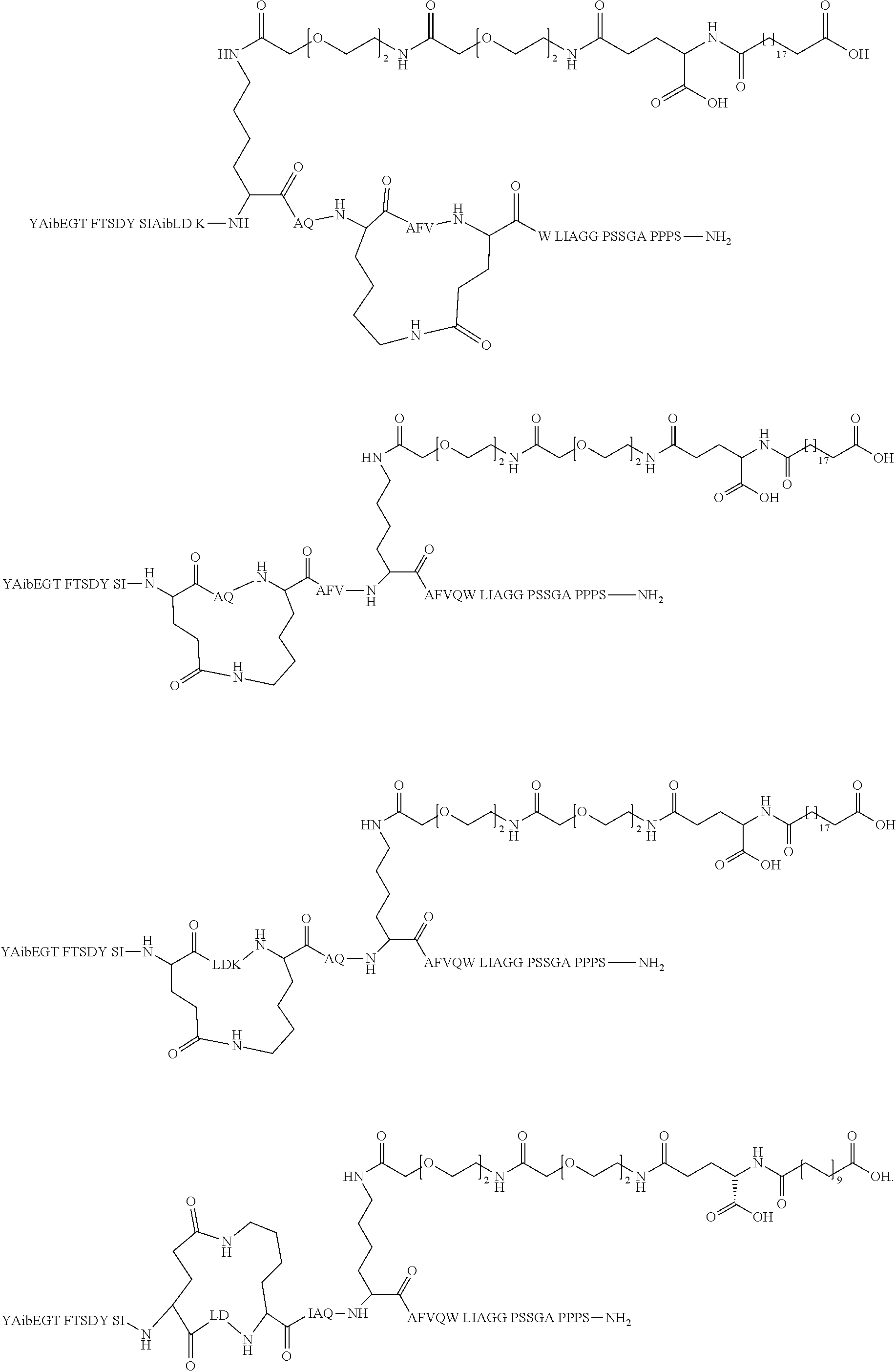

In some embodiments of the present disclosure, provided is a use of the compound or the pharmaceutically acceptable salt thereof in the manufacture of a medicament for treating obesity and diabetes.

The present disclosure also provides the following test method:

### Experimental Example: Inhibition Test of hERG Potassium Channels

A. Main Material

CHO-hERG cell line (Chinese hamster ovary cells stably expressing hERG channels), constructed in-house at Shanghai Institute of Materia Medica, Chinese Academy of Sciences; patch clamp amplifier Axopatch 200B, TASER® International.

B. Method

a) Cell Culture

CHO cells stably expressing hERG are cultured in cell culture dishes with a diameter of 35 mm, placed in an incubator with 5% CO₂ at 37° C., and passaged at a ratio of 1:5 every 48 hours. Culture medium formulation: 90% F12 (Invitrogen), 10% fetal bovine serum (Gibco), 100 μg/mL G418 (Invitrogen) and 100 μg/mL Hygromycin B (Invitrogen). On the day of the test, the cell culture medium was aspirated, rinsed once with extracellular fluid, and then 0.25% Trypsin-EDTA (Invitrogen) solution was added, followed by digestion at room temperature for 3 to 5 minutes. After the digestion solution was aspirated, the cells were resuspended with extracellular fluid, and transferred to a laboratory dish for electrophysiological recording for further use.

b) Preparation of Intracellular and Extracellular Fluid

Extracellular fluid needs to be prepared once a month. Intracellular fluid needs to be subpackaged and frozen at −20° C.

Extracellular fluid (mM): 145 NaCl, 4 KCl, 2 CaCl₂, 1 MgCl₂, 10 Glucose and 10 HEPES, adjusted to pH=7.4 with NaOH, with an osmotic pressure of 295 mOsm.

Intracellular fluid (mM): 120 KCl, 31.25 KOH, 5.374 CaCl₂), 1.75 MgCl₂, 4 Na₂ATP, 10 HEPES and 10 EGTA, adjusted to pH=7.2 with KOH, with an osmotic pressure of 285 mOsm.

c) Preparation of Compounds

The compound is dissolved in DMSO into a 20 mM stock solution. On the day of the test, the compound stock solution is serially 3-fold diluted with DMSO, i.e., 10 μL of the compound stock solution is added to 20 μL of DMSO to obtain 6 intermediate concentrations of the compound serially diluted with DMSO, respectively. The intermediate concentrations are 20, 6.66, 2.22, 0.74, 0.24 and 0.082 mM, respectively. Then 10 μL of the intermediate concentration of the compound is taken and added to 4990 μL of extracellular fluid, and 500-fold diluted to obtain the final concentration to be tested. The highest test concentration is 40 μM, which are 40, 13.3, 4.44, 1.48, 0.49 and 0.16 μM, respectively. The content of DMSO in the final concentration to be tested does not exceed 0.2%, and this concentration of DMSO has no effect on hERG potassium channels.

d) Electrophysiological Recording Process

In CHO (Chinese Hamster Ovary) cells stably expressing hERG potassium channels, hERG potassium channel current is recorded by whole cell patch clamp technique at room temperature. The glass microelectrode is made of a glass electrode filament (BF 150-86-10, Sutter) pulled by a glass microelectrode puller. The tip resistance after pouring the electrode internal solution into the electrode is about 2-5 MS/. The glass microelectrode is inserted into the amplifier probe to connect to an Axopatch 200B (Molecular Devices) patch clamp amplifier. The clamping potential and data record are controlled and recorded by the computer with pClamp 10 software, and the sampling frequency is 10 kHz, and the wave filtering frequency is 2 kHz. After whole-cell recordings are obtained, the cells are clamped at −80 mV, and the step voltage of hERG potassium current (1 hERG) is evoked from −80 mV to +20 mV with a depolarizing voltage for 2 s, then repolarized to −50 mV, and returned to −80 mV after 1 s. This voltage stimulation is given every 10 s, and the administration process is started after confirming that the hERG potassium current is stable (1 min). Serial administration is started from a low test concentration of the compounds, with each test concentration administered for at least 1 minute. At least 3 cells (n>3) are tested for each concentration of the compounds, and at least 2 cells (n>2) are tested for each concentration of the positive compounds.

e) Data Analysis

In each complete current recording, the inhibition percentage for each compound effect concentration can be calculated based on the percentage of peak current in the negative control. The dose-effect relationship curve is obtained by fitting the standard Hill equation, and the specific equation is as follows:

I_{(C)}=I_{b}+(I_{fr}−I_{b})*cⁿ/(IC₅₀ⁿ+cⁿ)
c is the compound test concentration, n is the slope

Both curve fitting and inhibition rate calculation are analyzed and completed by Qpatch analysis software. If the inhibition rate at the lowest concentration exceeds the half inhibition rate or the inhibition rate at the highest concentration does not reach the half inhibition rate, the corresponding IC₅₀ of the compound is lower than the lowest concentration or the IC₅₀ value is greater than the highest concentration.

C. Test Results and Conclusions

The compounds in the present disclosure have no hERG-related risks.

### Experimental Example: Inhibition for Cytochrome P450 Isoenzyme

A. Experimental Purpose

To determine the inhibitory effect of the test compounds on the activity of human liver microsomal cytochrome P450 isozymes (CYP1A2, CYP2C9, CYP2C19, CYP2D6 and CYP3A4).

B. Experimental Operation

First, the test compound (10 mM) is gradient diluted to prepare a working solution (100×final concentration). Concentrations of the working solution are 5 mM, 1.5 mM, 0.5 mM, 0.15 mM, 0.05 mM, 0.015 mM and 0.005 mM, respectively. At the same time, positive inhibitors of the P450 isozymes (CYP1A2, CYP2C9, CYP2C19, CYP2D6 and CYP3A4) and the working solution of the specific substrate mixture thereof are prepared; human liver microsomes frozen in a −80° C. refrigerator are thawed on ice. Once the human liver microsomes are completely dissolved, the solution is diluted with PBS (phosphate buffer solution) to prepare a working solution with a certain concentration (0.253 mg/mL); 20 IL of substrate mixture solution is added to a reaction plate (20 μL of PB is added to the Blank well). At the same time, 158 μL of human liver microsome working solution is added to the reaction plate, and the reaction plate is placed on ice for use; at this time, 2 μL of the test compound (N=1) and positive inhibitors (N=2) of each concentration are added to the corresponding well, and the group without inhibitors (the test compound or positive inhibitor) is added with the corresponding organic solvent, as the sample of the control group; after pre-incubating in a 37° C. water bath for 10 minutes, 20 μL of NADPH solution is added to the reaction plate, and incubated in a 37° C. water bath for 10 minutes; 400 μL of cold acetonitrile solution is added to terminate the reaction; the reaction plate is placed on a shaker and shaken for 10 minutes; the mixture is centrifuged at 4,000 rpm for 20 minutes; 200 μL of the supernatant is taken and added to 100 μL of water to dilute the sample; finally the reaction plate is sealed and shaken up for LC/MS/MS detection.

C. Experimental Results and Conclusions

The compounds in the present disclosure have no CYP inhibition-related risks.

Technical Effect

The compounds in the present disclosure have strong agonist activity on GLP-1R/GIPR; the compounds in the present disclosure have excellent pharmacokinetic properties; the compounds in the present disclosure have excellent plasma stability; the compounds in the present disclosure have very high plasma-protein binding; the compounds in the present disclosure have excellent in vivo efficacy.

### Definition and Description

Unless otherwise specified, the following terms and phrases used herein have the following meanings. A specific term or phrase should not be considered indefinite or unclear in the absence of a particular definition, but should be understood according to the common meaning. When a trade name appears herein, it is intended to refer to its corresponding commercial product or active ingredient thereof.

The term “pharmaceutically acceptable” is used herein in terms of those compounds, materials, compositions, and/or dosage forms, which are suitable for use in contact with human and animal tissues within the scope of reliable medical judgment, without excessive toxicity, irritation, anaphylactic reaction or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting the compound with a sufficient amount of a base in a pure solution or a suitable inert solvent. The pharmaceutically acceptable base addition salt includes a salt of sodium, potassium, calcium, ammonium, organic amine or magnesium, or similar salts. When the compound of the present disclosure contains a relatively basic functional group, an acid addition salt can be obtained by contacting the compound with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and salts of amino acid (such as arginine and the like), and a salt of an organic acid such as glucuronic acid and the like. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

As used herein, A or Ala represents alanine, and has a structure of

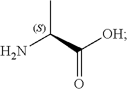

R or Arg represents arginine, and has a structure of

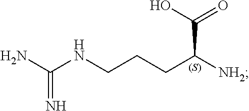

N or Asn represents asparagine, and has a structure of

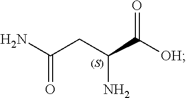

D or Asp represents aspartic

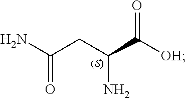

C or Cys represents cysteine, and has a acid, and has a structure of

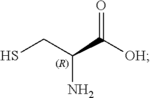

Q or Gln represents glutamine, and has a structure of structure of

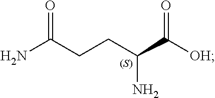

E or Glu represents glutamic acid, and has a structure of

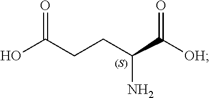

G or Gly represents glycine, and has a structure of

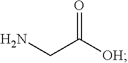

H or His represents histidine, and has a structure of

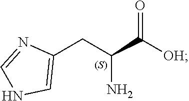

I or Ile represents isoleucine, and has a structure of

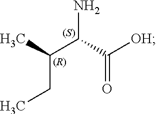

L or Leu represents leucine, and has a structure of

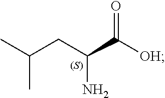

K or Lys represents lysine, and has a structure of

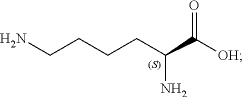

M or Met represents methionine, and has a structure of

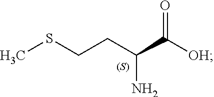

F or Phe represents phenylalanine, and has a structure of

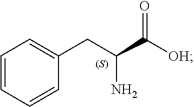

P or Pro represents proline, and has a structure of

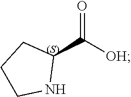

S or Ser represents serine, and has a structure of

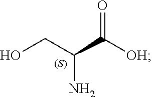

T or Thr represents threonine, and has a structure of

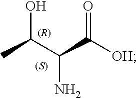

W or Trp represents tryptophan, and has a structure of

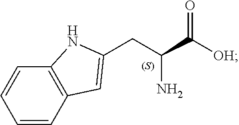

Y or Tyr represents tyrosine, and has a structure of

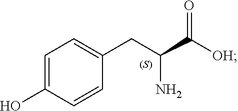

V or Val represents valine, and has a structure of

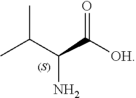

The term “treating” includes inhibiting, slowing, stopping or reversing the progression or severity of an existing symptom or disease.

Unless otherwise specified, the term “isomer” is intended to include a geometric isomers, a cis-trans isomer, a stereoisomer, an enantiomer, an optical isomer, diastereoisomers and a tautomeric isomer.

The compounds of the present disclosure may exist in specific geometric or stereoisomeric forms. The present disclosure contemplates all such compounds, including cis and Trans isomers, (−)-and (+)-enantiomers, (R)-and (S)-enantiomers, diastereoisomers, (D)-isomers, (L)-isomers, and racemic and other mixtures thereof, such as enantiomers or diastereomer enriched mixtures, all of which are within the scope of the present disclosure. Additional asymmetric carbon atoms may be present in substituents such as alkyl. All these isomers and their mixtures are encompassed within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) or a dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) and a straight dashed bond ( [symbol] ).

Unless otherwise specified, the terms “enriched in one isomer”, “enriched in isomers”, “enriched in one enantiomer” or “enriched in enantiomers” refer to the content of one of the isomers or enantiomers is less than 100%, and the content of such isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomers, as well as D and L isomers, can be prepared by chiral synthesis or chiral reagents or other conventional techniques. If one enantiomer of a compound of the present disclosure is desired, the pure desired enantiomer can be prepared by asymmetric synthesis or derivatization of chiral auxiliary followed by separating the resulting diastereomeric mixture and cleaving the auxiliary group. Alternatively, when the molecule contains a basic functional group (such as an amino) or an acidic functional group (such as a carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereoisomer are generally isolated through chromatography which uses a chiral stationary phase and optionally combines with a chemical derivative method (such as carbamate generated from amine).

The compound of the present disclosure may contain an unnatural proportion of atomic isotope at one or more atom(s) that constitute the compound. For example, the compound can be labeled with a radioactive isotope, such as tritium (³H), iodine-125(¹²⁵I) or C-14 (¹⁴C). For another example, deuterated drugs can be formed by replacing hydrogen with heavy hydrogen, the bond formed by deuterium and carbon is stronger than that by ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

Unless otherwise specified, the term “C₁₋₃ alkyl” refers to a linear or branched saturated hydrocarbon group consisting of 1 to 3 carbon atoms. The C₁₋₃ alkyl includes C₁₋₂ and C₂₋₃ alkyl, etc.; it can be monovalent (such as methyl), divalent (such as methylene) or multivalent (such as methine). Examples of C₁₋₃ alkyl include but are not limited to methyl (Me), ethyl (Et), propyl (including n-propyl and isopropyl), etc.

Unless otherwise specified, “S—NH₂” in the compounds of the present disclosure is used to indicate the replacement of the carboxyl on serine with an amide.

The structure of the compounds of the present disclosure can be confirmed by conventional methods known to those skilled in the art, and if the disclosure involves an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: φ/ω scan, and after collecting the relevant data, the crystal structure can be further analyzed by direct method (Shelxs97).

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred implementations include but are not limited to the examples of the present disclosure.

The solvents used in the present disclosure are commercially available.

The present disclosure uses the following abbreviations: “aq” represents water; “eq” represents equivalent; “DCM” represents dichloromethane; “PE” represents petroleum ether; “DMSO” represents dimethyl sulfoxide; “MeOH” represents methanol; “BOC” represents tert-butoxycarbonyl, which is an amine protecting group; “r.t.” represents room temperature; “O/N” represents overnight; “THF” represents tetrahydrofuran; “Boc₂O” represents di-tert-butyl dicarbonate; “TFA” represents trifluoroacetic acid; “DIEA” represents diisopropylethylamine; “DMF” represents N,N-dimethylformamide; “HBTU” represents 0-(benzotriazol-1-yl)-N,N,N′,N′-tetramethyluronium hexafluorophosphate; “HOBT” represents 1-hydroxybenzotriazole; “HOAT” represents 1-hydroxy-7-azabenzotriazole; “DIC” represents N,N′-diisopropylcarbodiimide; “DBU” represents 1,8-diazabicyclo[5.0.4]undec-7-ene; “PhSiH” represents phenylsilane; “Pd(PPh₃)₄” represents tetrakis(triphenylphosphine)palladium.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and specific embodiments thereof have also been disclosed; for those skilled in the art, it is obvious to make various modifications and improvements to the embodiments of the present disclosure without departing from the spirit and scope of the present disclosure.

### Intermediate B-1

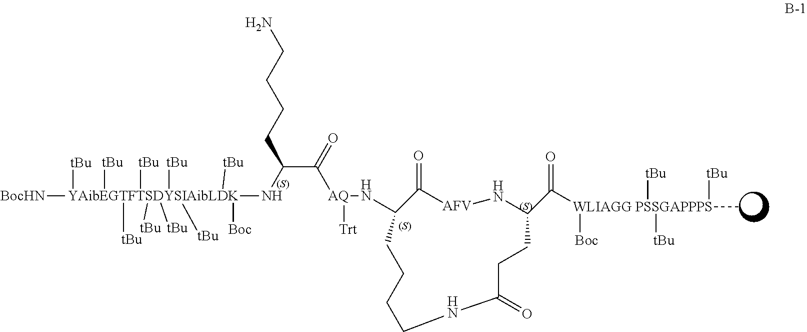

Step 1: 1.1 g of 4-(2′,4′-dimethoxyphenyl-Fmoc-aminmethyl)-phenoxyacetamido-methylbenzhydryl amine resin (loading value, i.e., Sub=0.28 mmol/g) was weighed and added into a reaction column, and then DMF (50 mL) was added to the reaction column, bubbled with nitrogen for 2 hours. The waste was drained until no liquid flowed out, and the resin was washed with DMF (50 mL) 5 times (1 minute each time), and the waste was drained until no liquid flowed out.

Step 2: 20% of piperidine/DMF (50 mL) was added to the reaction column, bubbled with nitrogen for 20 minutes, and then the waste was drained until no liquid flowed out. The resin was washed with DMF (50 mL) 5 times (1 minute each time), and the waste was drained until no liquid flowed out. The resin was tested with ninhydrin and showed blue.

Step 3: Coupling of Amino Acids

3.1 Coupling of Fmoc-Ser(tBu)-OH

1. Fmoc-Ser(tBu)-OH (3.0 eq) was weighed and added to the above resin, and DIEA (6.00 eq) plus 10 mL of DMF were added to the reaction column, bubbled with nitrogen, and HBTU (2.85 eq) was added after the amino acid was dissolved. The nitrogen was adjusted to make the resin bulge evenly.

2. The reaction was carried out for 0.5 hours at 25° C. The resin was tested with ninhydrin and then showed colorless and transparent.

3. The reaction solution was removed, and the resin was washed with DMF 5 times (50 mL each time, 1 min each time). The waste was drained until no liquid flowed out.

3.2 Coupling of Fmoc-Pro-OH

1. 20% of piperidine/DMF (50 mL) was added to the reaction column, bubbled with nitrogen for 20 minutes, and then the waste was drained until no liquid flowed out. The resin was washed with DMF (50 mL) 5 times (1 minute each time), and the waste was drained until no liquid flowed out. The resin was tested with ninhydrin and showed blue.

2. Fmoc-Pro-OH (3.0 eq) was weighed and added to the above resin, and DIEA (6.00 eq) plus 10 mL of DMF were added to the reaction column, bubbled with nitrogen, and HBTU (2.85 eq) was added after the amino acid was dissolved. The nitrogen was adjusted to make the resin bulge evenly.

3. The reaction was carried out for 0.5 hours at 25° C. The resin was tested with ninhydrin and then showed colorless and transparent.

4. The reaction solution was removed, and the resin was washed with DMF 5 times (50 mL each time, 1 min each time). The waste was drained until no liquid flowed out.

Step 3.2 was Repeated to Complete the Coupling of the Following Amino Acids

| NO. | Raw Material | Coupling Reagent |
|---|---|---|
| 3.3 | Fmoc—Pro-OH (3.00 eq) | HBTU (2.85 eq) and DIEA (6.00 eq) |
| 3.4 | Fmoc—Pro-OH (3.00 eq) | HBTU (2.85 eq) and DIEA (6.00 eq) |
| 3.5 | Fmoc—Ala-OH (3.00 eq) | HBTU (2.85 eq) and DIEA (6.00 eq) |
| 3.6 | Fmoc—Gly-OH (3.00 eq) | HBTU (2.85 eq) and DIEA (6.00 eq) |
| 3.7 | Fmoc—Ser(tBu)-OH | HBTU (2.85 eq) and DIEA (6.00 eq) |
| | (3.00 eq) | |
| 3.8 | Fmoc—Ser(tBu)-OH | HBTU (2.85 eq) and DIEA (6.00 eq) |
| | (3.00 eq) | |
| 3.9 | Fmoc—Pro-OH (3.00 eq) | HBTU (2.85 eq) and DIEA (6.00 eq) |
| 3.10 | Fmoc—Gly-OH (3.00 eq) | HBTU (2.85 eq) and DIEA (6.00 eq) |
| 3.11 | Fmoc—Gly-OH (3.00 eq) | HBTU (2.85 eq) and DIEA (6.00 eq) |
| 3.12 | Fmoc—Ala-OH (3.00 eq) | HBTU (2.85 eq) and DIEA (6.00 eq) |
| 3.13 | Fmoc—Ile-OH (3.00 eq) | HBTU (2.85 eq) and DIEA (6.00 eq) |
| 3.14 | Fmoc—Leu-OH (3.00 eq) | HBTU (2.85 eq) and DIEA (6.00 eq) |
| 3.15 | Fmoc—Trp(Boc) OH | HBTU (2.85 eq) and DIEA (6.00 eq) |
| | (3.00 eq) | |
| 3.16 | Fmoc—Glu(OAll)-OH | HBTU (1.90 eq) and DIEA (4.00 eq) |
| | (2.00 eq) | |
| 3.17 | Fmoc—Val-OH (3.00 eq) | HBTU (2.85 eq) and DIEA (6.00 eq) |
| 3.18 | Fmoc—Phe-OH (3.00 eq) | HBTU (2.85 eq) and DIEA (6.00 eq) |
| 3.19 | Fmoc—Ala-OH (3.00 eq) | HBTU (2.85 eq) and DIEA (6.00 eq) |
| 3.20 | Fmoc—Lys(Alloc)-OH | HOBT (2.00 eq) and DIC (2.00 eq) |
| | (2.00 eq) | |
| 3.21 | Fmoc—Gln(Trt)-OH | HBTU (2.85 eq) and DIEA (6.00 eq) |
| | (3.00 eq) | |
| 3.22 | Fmoc—Ala-OH (3.00 eq) | HBTU (2.85 eq) and DIEA (6.00 eq) |
| 3.23 | Fmoc—Lys(Dde)-OH | HBTU (2.85 eq) and DIEA (6.00 eq) |
| | (3.00 eq) | |
| 3.24 | Fmoc—Lys(Boc)—OH | HBTU (2.85 eq) and DIEA (6.00 eq) |
| | (3.00 eq) | |
| 3.25 | Fmoc—Asp(tBu)-OH | HATU (5.70 eq) and DIEA (12.0 eq) |
| | (6.00 eq) | |
| 3.26 | Fmoc—Leu-OH (6.00 eq) | HATU (5.70 eq) and DIEA (12.0 eq) |
| 3.27 | Fmoc—Aib-OH (6.00 eq) | HATU (5.70 eq) and DIEA (12.0 eq) |
| 3.28 | Fmoc—Ile-OH (6.00 eq) | HOAT (6.00 eq) and DIC (6.00 eq) |
| 3.29 | Fmoc—Ser(tBu)-OH | HATU (5.70 eq) and DIEA (12.0 eq) |
| | (6.00 eq) | |
| 3.30 | Fmoc—Tyr(tBu)-OH | HATU (5.70 eq) and DIEA (12.0 eq) |
| | (6.00 eq) | |
| 3.31 | Fmoc—Asp(tBu)-OH | HATU (5.70 eq) and DIEA (12.0 eq) |
| | (6.00 eq) | |
| 3.32 | Fmoc—Ser(tBu)-OH | HATU (5.70 eq) and DIEA (12.0 eq) |
| | (6.00 eq) | |
| 3.33 | Fmoc—Thr(tBu)-OH | HATU (5.70 eq) and DIEA (12.0 eq) |
| | (6.00 eq) | |
| 3.34 | Fmoc—Phe-OH (6.00 eq) | HATU (5.70 eq) and DIEA (12.0 eq) |
| 3.35 | Fmoc—Thr(tBu)-OH | HATU (5.70 eq) and DIEA (12.0 eq) |
| | (6.00 eq) | |
| 3.36 | Fmoc—Gly-OH (6.00 eq) | HATU (5.70 eq) and DIEA (12.0 eq) |
| 3.37 | Fmoc—Glu(tBu)-OH | HOBT (6.00 eq) and DIC (6.00 eq) |
| | (6.00 eq) | |
| 3.38 | Fmoc—Aib-OH (3.00 eq) | HATU (2.85 eq) and DIEA (6.00 eq) |
| 3.39 | Boc Tyr(tBu)-OH | HATU (2.85 eq) and DIEA (6.00 eq) |
| | (3.00 eq) | |

3.40 Deprotection of Alloc and OAll

1. PhSiH₃ (20.0 eq) and DCM (10 mL) were added to the reaction column, after bubbling with nitrogen, Pd(PPh₃)₄ (0.2 eq) was added, and bubbled with nitrogen for 20 min. The reaction was carried out twice, and the waste was drained until no liquid flowed out.

2. The resin was washed with DMF 5 times (50 mL each time, 1 min each time). The waste was drained until no liquid flowed out.

3.41 Ring Closure of Amide

1. DIEA (3.00 eq) plus 10 mL of DMF were added to the reaction column, bubbled with nitrogen, and HATU (1.5 eq) was added after the amino acid was dissolved. The nitrogen was adjusted to make the resin bulge evenly.

2. The reaction was carried out for 0.5 hours at 25° C. The resin was tested with ninhydrin and then showed colorless and transparent.

3. The resin was washed with DMF 5 times (50 mL each time, 1 min each time). The waste was drained until no liquid flowed out.

3.42 Deprotection of Dde

1. 3% of hydrazine hydrate/DMF (50 mL) was added to the reaction column, bubbled with nitrogen for 15 minutes, and then the waste was drained until no liquid flowed out. The resin was washed with DMF 5 times (50 mL each time, 1 min each time) and the waste was drained until no liquid flowed out to obtain intermediate B-1. The resin was tested with ninhydrin and showed blue.

### Intermediate B-2

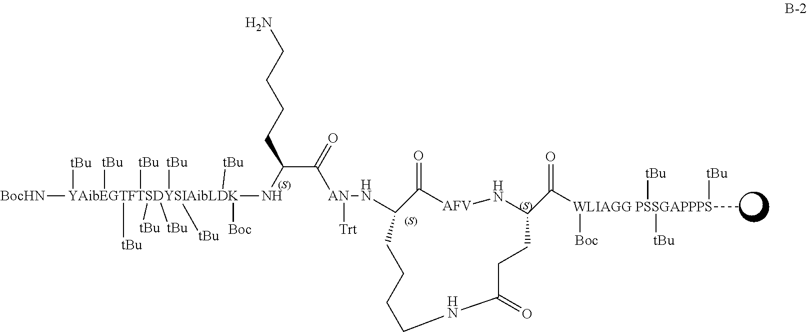

Referring to the synthesis method of B-1, B-2 was obtained after the following operations such as coupling of amino acids, deprotection of Alloc and OA11, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Glu(OAll)-OH | HBTU (1.90 eq) and DIEA (4.00 eq) |
| | (2.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Alloc)-OH | HOBT (2.00 eq) and DIC (2.00 eq) |
| | (2.00 eq) | |
| 21 | Fmoc—Asn(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Lys(Dde)-OH | HBTU (2.85 eq) and DIEA (6.00 eq) |
| | (3.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | HATU (5.70 eq) and DIEA (12.0 eq) |
| | (6.00 eq) | |
| 26 | Fmoc—Leu-OH (6.00 eq) | HATU (5.70 eq) and DIEA (12.0 eq) |
| 27 | Fmoc—Aib-OH (6.00 eq) | HATU (5.70 eq) and DIEA (12.0 eq) |
| 28 | Fmoc—Ile-OH (6.00 eq) | HOAT (6.00 eq) and DIC (6.00 eq) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(tBu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIBA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-3

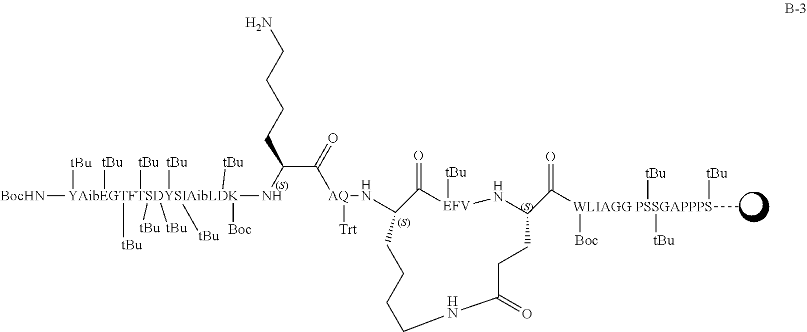

Referring to the synthesis method of B-1, B-3 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Glu(OAll)-OH | HBTU (1.90 eq) and DIEA (4.00 eq) |
| | (2.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Glu(tBu)-OH | HBTU (2.85 eq) and DIEA (6.00 eq) |
| | (3.00 eq) | |
| 20 | Fmoc—Lys(Alloc)-OH | HOBT (2.00 eq) and DIC (2.00 eq) |
| | (2.00 eq) | |
| 21 | Fmoc—Gln(Trt)-OH | HBTU (2.85 eq) and DIEA (6.00 eq) |
| | (3.00 eq) | |
| 22 | Fmoc—Ala-OH | HBTU (2.85 eq) and DIEA (6.00 eq) |
| | (3.00 eq) | |
| 23 | Fmoc—Lys(Dde)-OH | HBTU (2.85 eq) and DIEA (6.00 eq) |
| | (3.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | HATU (5.70 eq) and DIEA (12.0 eq) |
| | (6.00 eq) | |
| 26 | Fmoc—Leu-OH (6.00 eq) | HATU (5.70 eq) and DIEA (12.0 eq) |
| 27 | Fmoc—Aib-OH (6.00 eq) | HATU (5.70 eq) and DIEA (12.0 eq) |
| 28 | Fmoc—Ile-OH (6.00 eq) | HOAT (6.00 eq) and DIC (6.00 eq) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(tBu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-4

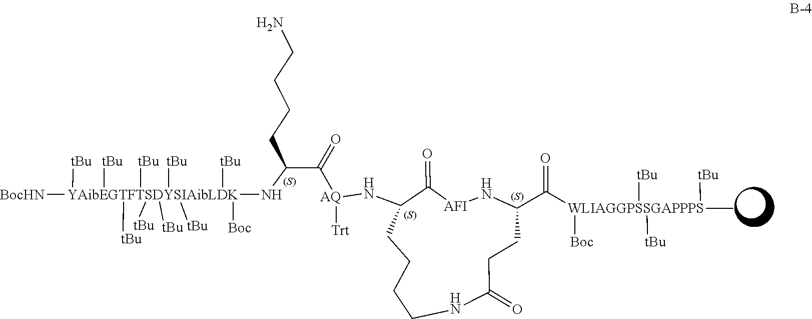

Referring to the synthesis method of B-1, B-4 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Glu(OAll)-OH | HBTU (1.90 eq) and DIEA (4.00 eq) |
| | (2.00 eq) | |
| 17 | Fmoc—Ile-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Alloc)-OH | HOBT (2.00 eq) and DIC (2.00 eq) |
| | (2.00 eq) | |
| 21 | Fmoc—Gln(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Lys(Dde)-OH | HBTU (2.85 eq) and DIEA (6.00 eq) |
| | (3.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | HATU (5.70 eq) and DIEA (12.0 eq) |
| | (6.00 eq) | |
| 26 | Fmoc—Leu-OH (6.00 eq) | HATU (5.70 eq) and DIEA (12.0 eq) |
| 27 | Fmoc—Aib-OH (6.00 eq) | HATU (5.70 eq) and DIEA (12.0 eq) |
| 28 | Fmoc—Ile-OH (6.00 eq) | HOAT (6.00 eq) and DIC (6.00 eq) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(tBu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-5

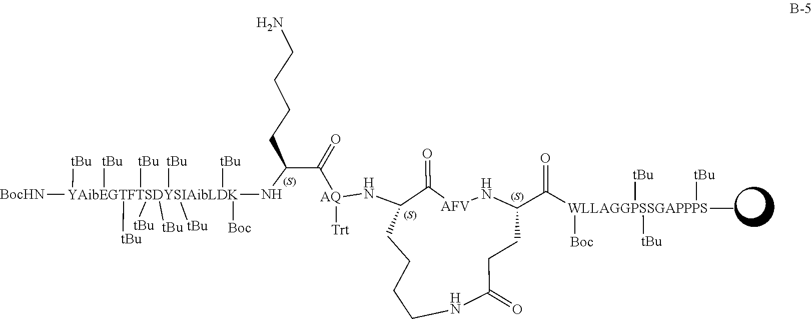

Referring to the synthesis method of B-1, B-5 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Leu-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Glu(OAll)-OH | HBTU (1.90 eq) and DIEA (4.00 eq) |
| | (2.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Alloc)-OH | HOBT (2.00 eq) and DIC (2.00 eq) |
| | (2.00 eq) | |
| 21 | Fmoc—Gln(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Lys(Dde)-OH | HBTU (2.85 eq) and DIEA (6.00 eq) |
| | (3.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | |
| | (6.00 eq) | HATU (5.70 eq) and DIEA (12.0 eq) |
| 26 | Fmoc—Leu-OH (6.00 eq) | HATU (5.70 eq) and DIEA (12.0 eq) |
| 27 | Fmoc—Aib-OH (6.00 eq) | HATU (5.70 eq) and DIEA (12.0 eq) |
| 28 | Fmoc—Ile-OH (6.00 eq) | HOAT (6.00 eq) and DIC (6.00 eg) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(tBu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-6

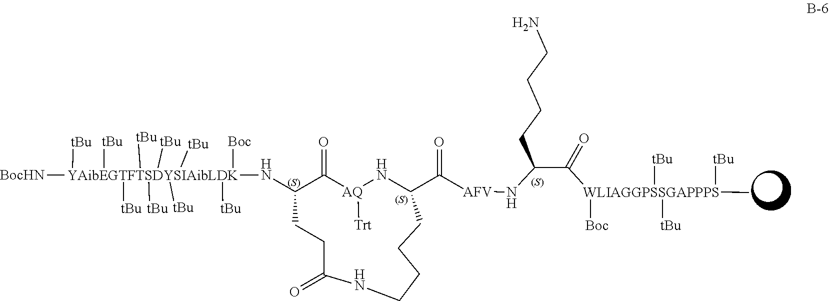

Referring to the synthesis method of B-1, B-6 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Lys(Dde)-OH | HBTU (1.90 eq) and DIEA (4.00 eq) |
| | (3.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Alloc)-OH | HOBT (2.00 eq) and DIC (2.00 eq) |
| | (2.00 eq) | |
| 21 | Fmoc—Gln(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Glu(OAll)-OH | HBTU (2.85 eq) and DIEA (6.00 eq) |
| | (2.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | HATU (5.70 eq) and DIEA (12.0 eq) |
| | (6.00 eq) | |
| 26 | Fmoc—Leu-OH (6.00 eq) | HATU (5.70 eq) and DIEA (12.0 eq) |
| 27 | Fmoc—Aib-OH (6.00 eq) | HATU (5.70 eq) and DIEA (12.0 eq) |
| 28 | Fmoc—Ile-OH | HOAT (6.00 eq) and DIC (6.00 eq) |
| | (6.00 eq) | |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(tBu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-7

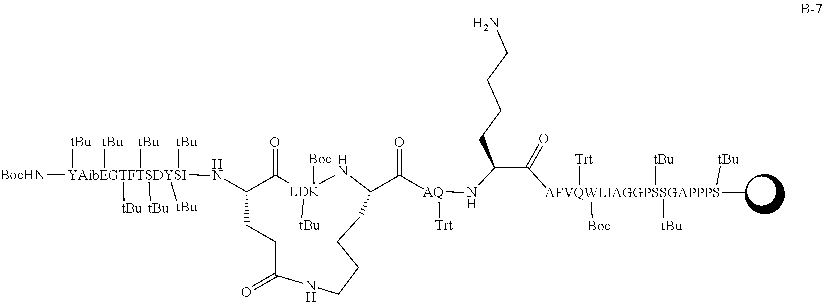

Referring to the synthesis method of B-1, B-7 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Gln(Trt)-OH | HBTU (1.90 eq) and DIEA (4.00 eq) |
| | (2.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Dde)-OH | HOBT (2.00 eq) and DIC (2.00 eq) |
| | (3.00 eq) | |
| 21 | Fmoc—Gln(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Lys(Alloc)-OH | HBTU (2.85 eq) and DIEA (6.00 eq) |
| | (2.00 eq) | |
| 24 | Fmoc—Lys(Boc) OH | HBTU (2.85 eq) and DIEA (6.00 eq) |
| | (3.00 eq) | |
| 25 | Fmoc—Asp(tBu)-OH | HATU (5.70 eq) and DIEA (12.0 eq) |
| | (6.00 eq) | |
| 26 | Fmoc—Leu-OH (6.00 eq) | HATU (5.70 eq) and DIEA (12.0 eq) |
| 27 | Fmoc—Glu(OAII)-OH | HATU (5.70 eq) and DIEA (12.0 eq) |
| | (2.00 eq) | |
| 28 | Fmoc—Ile-OH (6.00 eq) | HOAT (6.00 eq) and DIC (6.00 eq) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIEA (12.0 eq) |
| | (6.00 eq) | |
| 31 | Fmoc—Asp(Bu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIBA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Intermediate B-8

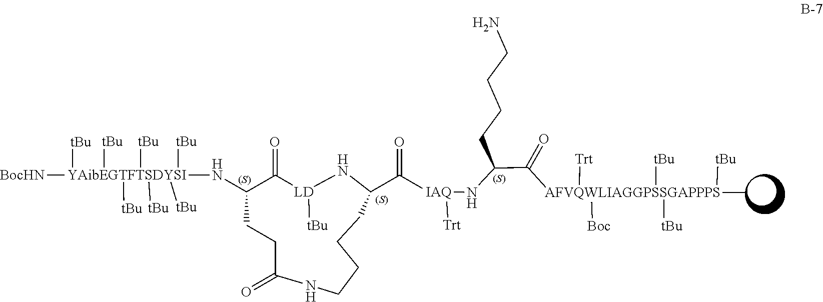

Referring to the synthesis method of B-1, B-8 was obtained after the following operations such as coupling of amino acids, deprotection of Alloc and OAll, ring closure of amide, and deprotection of Dde.

| NO. | Raw Material | Coupling Reagent |
|---|---|---|
| 1 | Fmoc—Ser(tBu)-OH | HBTU (2.85 eq) and DIEA (6.00 eq) |
| | (3.00 eq) | |
| 2 | Fmoc—Pro-OH (3.00 eq) | HBTU (2.85 eq) and DIEA (6.00 eq) |
| 3 | Fmoc—Pro-OH (3.00 eq) | HBTU (2.85 eq) and DIEA (6.00 eq) |
| 4 | Fmoc—Pro-OH (3.00 eq) | HBTU (2.85 eq) and DIEA (6.00 eq) |
| 5 | Fmoc—Ala-OH (3.00 eq) | HBTU (2.85 eq) and DIEA (6.00 eq) |
| 6 | Fmoc—Gly-OH (3.00 eq) | HBTU (2.85 eq) and DIEA (6.00 eq) |
| 7 | Fmoc—Ser(tBu)-OH | HBTU (2.85 eq) and DIEA (6.00 eq) |
| | (3.00 eq) | |
| 8 | Fmoc—Ser(tBu)-OH | HBTU (2.85 eq) and DIEA (6.00 eq) |
| | (3.00 eq) | |
| 9 | Fmoc—Pro-OH (3.00 eq) | HBTU (2.85 eq) and DIEA (6.00 eq) |
| 10 | Fmoc—Gly-OH (3.00 eq) | HBTU (2.85 eq) and DIEA (6.00 eq) |
| 11 | Fmoc—Gly-OH (3.00 eq) | HBTU (2.85 eq) and DIEA (6.00 eq) |
| 12 | Fmoc—Ala-OH (3.00 eq) | HBTU (2.85 eq) and DIEA (6.00 eq) |
| 13 | Fmoc—Ile-OH (3.00 eq) | HBTU (2.85 eq) and DIEA (6.00 eq) |
| 14 | Fmoc—Leu-OH (3.00 eq) | HBTU (2.85 eq) and DIEA (6.00 eq) |
| 15 | Fmoc—Trp(Boc) OH | HBTU (2.85 eq) and DIEA (6.00 eq) |
| | (3.00 eq) | |
| 16 | Fmoc—Gln(Trt)-OH | HBTU (1.90 eq) and DIEA (4.00 eq) |
| | (2.00 eq) | |
| 17 | Fmoc—Val-OH (3.00 eq) | HBTU (2.85 eq) and DIEA (6.00 eq) |
| 18 | Fmoc—Phe-OH (3.00 eq) | HBTU (2.85 eq) and DIEA (6.00 eq) |
| 19 | Fmoc—Ala-OH (3.00 eq) | HBTU (2.85 eq) and DIEA (6.00 eq) |
| 20 | Fmoc—Lys(Dde)-OH | HOBT (2.00 eq) and DIC (2.00 eq) |
| | (3.00 eq) | |
| 21 | Fmoc—Gln(Trt)-OH | HBTU (2.85 eq) and DIEA (6.00 eq) |
| | (3.00 eq) | |
| 22 | Fmoc—Ala-OH (3.00 eq) | HBTU (2.85 eq) and DIEA (6.00 eq) |
| 23 | Fmoc—Ile-OH (3.00 eq) | HBTU (2.85 eq) and DIEA (6.00 eq) |
| 24 | Fmoc—Lys(Alloc)-OH | HBTU (2.85 eq) and DIEA (6.00 eq) |
| | (2.00 eq) | |
| 25 | Fmoc—Asp(tBu)-OH | |
| | (6.00 eq) | HATU (5.70 eq) and DIEA (12.0 eq) |
| 26 | Fmoc—Leu-OH (6.00 eq) | HATU (5.70 eq) and DIEA (12.0 eq) |
| 27 | Fmoc—Glu(OAll)-OH | HATU (5.70 eq) and DIEA (12.0 eq) |
| | (2.00 eq) | |
| 28 | Fmoc—Ile-OH (6.00 eq) | HOAT (6.00 eq) and DIC (6.00 eq) |
| 29 | Fmoc—Ser(tBu)-OH | HATU (5.70 eq) and DIEA (12.0 eq) |
| | (6.00 eq) | |
| 30 | Fmoc—Tyr(tBu)-OH | HATU (5.70 eq) and DIBA (12.0 eq) |
| | (6.00 eq) | |
| 31 | Fmoc—Asp({Bu)-OH | HATU (5.70 eq) and DIEA (12.0 eq) |
| | (6.00 eq) | |
| 32 | Fmoc—Ser(tBu)-OH | HATU (5.70 eq) and DIEA (12.0 eq) |
| | (6.00 eq) | |
| 33 | Fmoc—Thr(tBu)-OH | HATU (5.70 eq) and DIEA (12.0 eq) |
| | (6.00 eq) | |
| 34 | Fmoc—Phe-OH (6.00 eq) | HATU (5.70 eq) and DIEA (12.0 eq) |
| 35 | Fmoc—Thr(tBu)-OH | HATU (5.70 eq) and DIEA (12.0 eq) |
| | (6.00 eq) | |
| 36 | Fmoc—Gly-OH (6.00 eq) | HATU (5.70 eq) and DIEA (12.0 eq) |
| 37 | Fmoc—Glu(tBu)-OH | HOBT (6.00 eq) and DIC (6.00 eq) |
| | (6.00 eq) | |
| 38 | Fmoc—Aib-OH (3.00 eq) | HATU (2.85 eq) and DIEA (6.00 eq) |
| 39 | Boc Tyr(tBu)-OH | HATU (2.85 eq) and DIEA (6.00 eq) |
| | (3.00 eq) | |

### Example 1

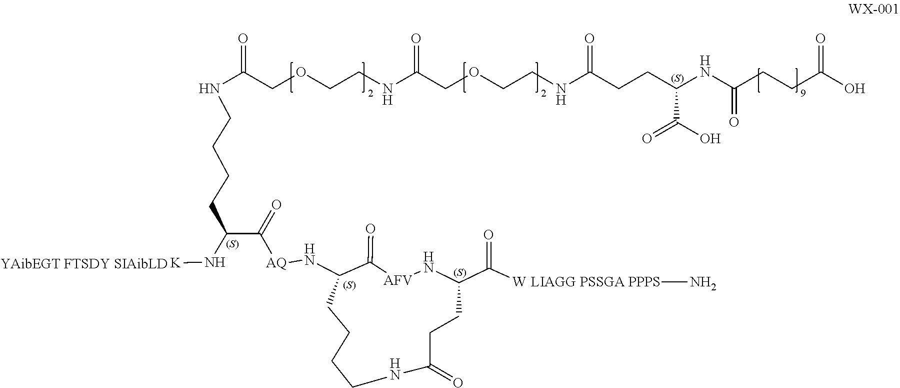

1. Coupling of long-chain fatty acids: Referring to synthesis step 3.2 of B-1 to complete the coupling of the following fragments:

| NO. | Raw Material | Coupling Reagent |
|---|---|---|
| 1 | Fmoc—AEEA-OH (3.00 eq) | HATU (2.85 eq) and DIEA (6.00 eq) |
| 2 | Fmoc—AEEA-OH (3.00 eq) | HATU (2.85 eq) and DIEA (6.00 eq) |
| 3 | Fmoc—Glu-OtBu (3.00 eq) | HATU (2.85 eq) and DIEA (6.00 eq) |
| 4 | 20-(tBu)-eicosanedioic acid | HATU (2.85 eq) and DIEA (6.00 eq) |
| | (3.00 eq) | |

2. Cleavage and Drying of Crude Peptide

2.1. The Cleavage Solution was Configured According to the Following Volume

| Reagent | Ratio |
|---|---|
| Trifluoroacetic acid | 91 |
| Triisopropylsilane | 3 |
| H₂O | 3 |
| 3-Mercaptopropionic acid | 3 |

The dried peptide resin was added to the prepared cleavage solution. The mixture was shaken on a shaker for 2.5 hours, and filtered, and the filtrate was added to ice isopropyl ether with 10 times the volume of the filtrate, centrifuged, and washed 3 times with isopropyl ether. After drying in vacuum for 2 hours, the crude peptide was obtained, and the polypeptide compound WX-001 was obtained after purification. The molecular weight of the polypeptide was confirmed by ESI-MS, calculated for 4811.4, found 4812.0.

### Example 2

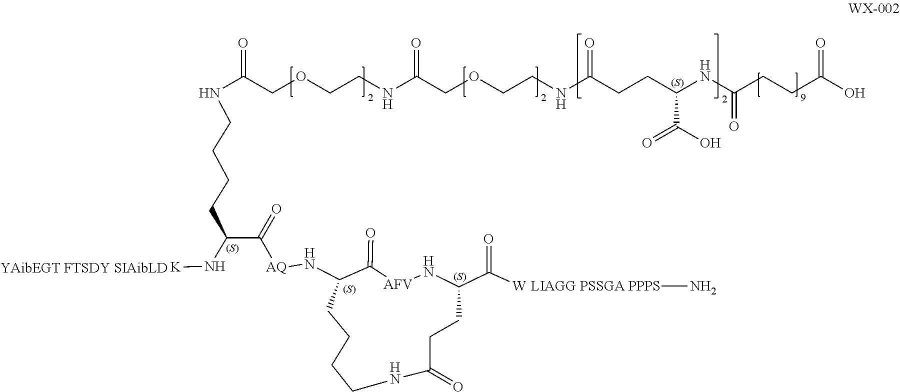

1. Coupling of Long-Chain Fatty Acids: Referring to Synthesis Step 3.2 of B-1 to Complete the Coupling of the Following Fragments:

| NO. | Raw Material | Coupling Reagent |
|---|---|---|
| 1 | Fmoc—AEEA-OH (3.00 eq) | HATU (2.85 eq) and DIEA (6.00 eq) |
| 2 | Fmoc—AEEA-OH (3.00 eq) | HATU (2.85 eq) and DIEA (6.00 eq) |
| 3 | Fmoc—Glu-OtBu (3.00 eq) | HATU (2.85 eq) and DIEA (6.00 eq) |
| 4 | Fmoc—Glu-OtBu (3.00 eq) | HATU (2.85 eq) and DIBA (6.00 eq) |
| 5 | 20-(tBu)-eicosanedioic | HATU (2.85 eq) and DIEA (6.00 eq) |
| | acid (3.00 eq) | |

2. Cleavage and Drying of Crude Peptide

2.1. The Cleavage Solution was Prepared According to the Following Volume

| Reagent | Ratio |
|---|---|
| Trifluoroacetic acid | 91 |
| Triisopropylsilane | 3 |
| H₂O | 3 |
| 3-Mercaptopropionic acid | 3 |

The dried peptide resin was added to the prepared cleavage solution. The mixture was shaken on a shaker for 2.5 hours, and filtered, and the filtrate was added to ice isopropyl ether with 10 times the volume of the filtrate, centrifuged, and washed 3 times with isopropyl ether. After drying in vacuum for 2 hours, the crude peptide was obtained, and the polypeptide compound WX-002 was obtained after purification. The molecular weight of the polypeptide was confirmed by ESI-MS, calculated for 4940.5, found 4940.6.

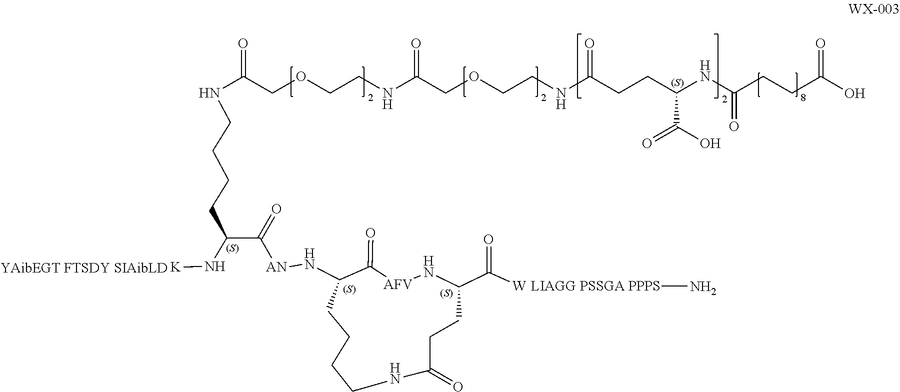

1. Coupling of Long-Chain Fatty Acids: Referring to Synthesis Step 3.2 of B-1 to Complete the Coupling of the Following Fragments:

| NO. | Raw Material | Coupling Reagent |
|---|---|---|
| 1 | Fmoc—AEEA-OH (3.00 eq) | HATU (2.85 eq) and DIEA (6.00 eq) |
| 2 | Fmoc—AEEA-OH (3.00 eq) | HATU (2.85 eq) and DIEA (6.00 eq) |
| 3 | Fmoc—Glu-OtBu (3.00 eq) | HATU (2.85 eq) and DIEA (6.00 eq) |
| 4 | Fmoc—Glu-OtBu (3.00 eq) | HATU (2.85 eq) and DIEA (6.00 eq) |
| 5 | 20-(tBu)-octadecanedioic | HATU (2.85 eq) and DIEA (6.00 eg) |
| | acid (3.00 eq) | |

2. Cleavage and Drying of Crude Peptide

2.1. The Cleavage Solution was Configured According to the Following Volume

| Reagent | Ratio |
|---|---|
| Trifluoroacetic acid | 91 |
| Triisopropylsilane | 3 |
| H₂O | 3 |
| 3-Mercaptopropionic acid | 3 |

The dried peptide resin was added to the prepared cleavage solution. The mixture was shaken on a shaker for 2.5 hours, and filtered, and the filtrate was added to ice isopropyl ether with 10 times the volume of the filtrate, centrifuged, and washed 3 times with isopropyl ether. After drying in vacuum for 2 hours, the crude peptide was obtained, and the polypeptide compound WX-003 was obtained after purification. The molecular weight of the polypeptide was confirmed by ESI-MS, calculated for 4912.5, found 4913.1.

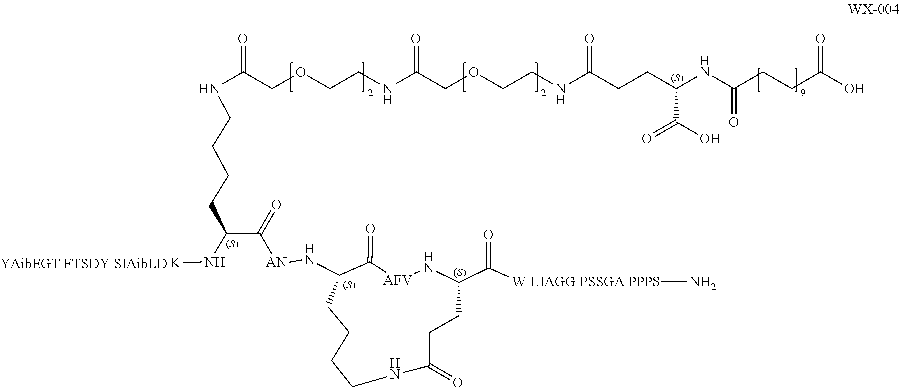

Referring to the synthesis of WX-001, WX-004 was obtained through intermediate B-2. The molecular weight of the polypeptide was confirmed by ESI-MS, calculated for 4797.4, found 4797.3.

### Example 5

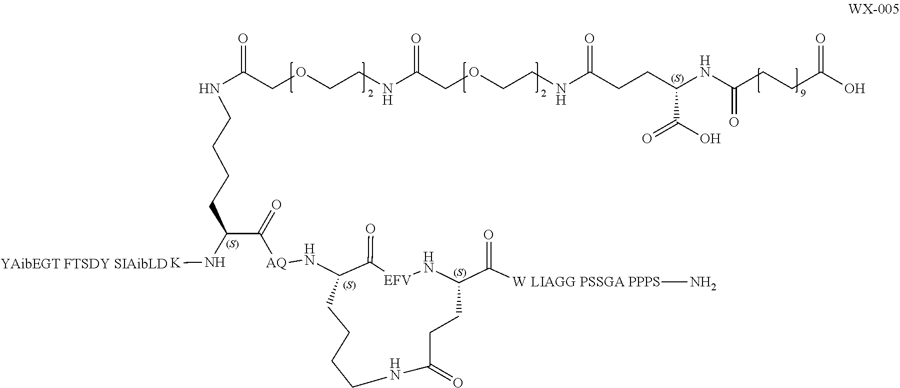

Referring to the synthesis of WX-001, WX-005 was obtained through intermediate B-3. The molecular weight of the polypeptide was confirmed by ESI-MS, calculated for 4869.5, found 4869.3.

### Example 6

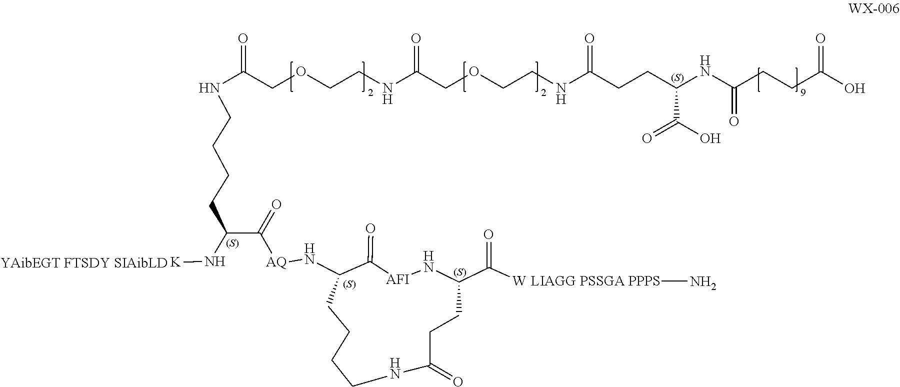

Referring to the synthesis of WX-001, WX-006 was obtained through intermediate B-4. The molecular weight of the polypeptide was confirmed by ESI-MS, calculated for 4825.5, found 4825.5.

### Example 7

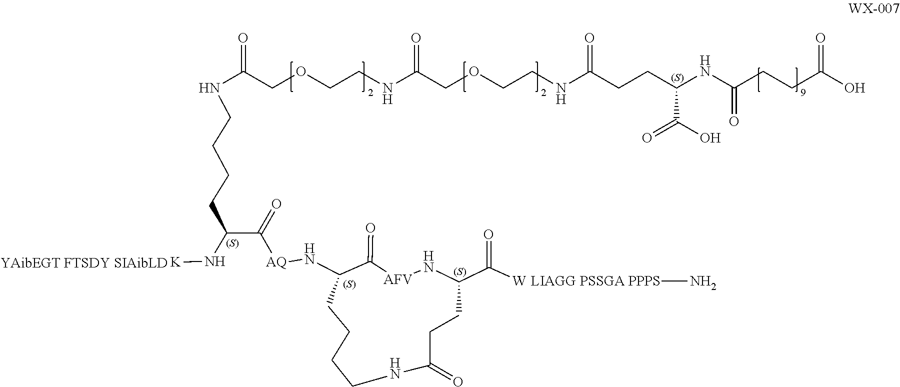

Referring to the synthesis of WX-001, WX-007 was obtained through intermediate B-5. The molecular weight of the polypeptide was confirmed by ESI-MS, calculated for 4811.4, found 4811.1.

### Example 8

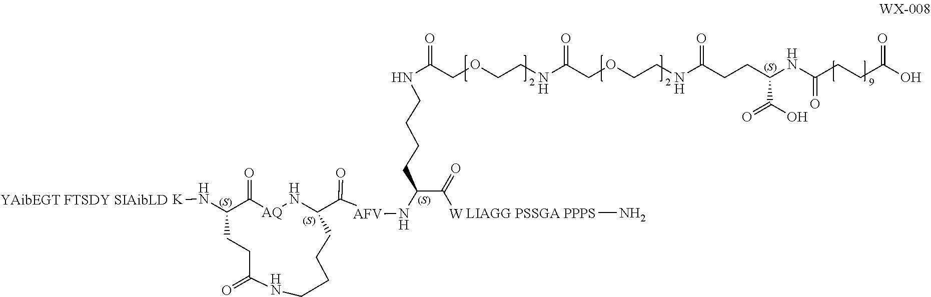

Referring to the synthesis of WX-001, WX-008 was obtained through intermediate B-6. The molecular weight of the polypeptide was confirmed by ESI-MS, calculated for 4811.4, found 4811.4.

### Example 9

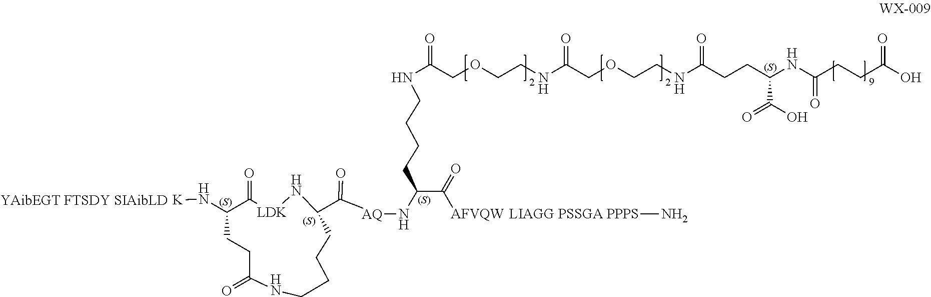

Referring to the synthesis of WX-001, WX-009 was obtained through intermediate B-7. The molecular weight of the polypeptide was confirmed by ESI-MS, calculated for 4854.5. found 4854.9.

### Example 10

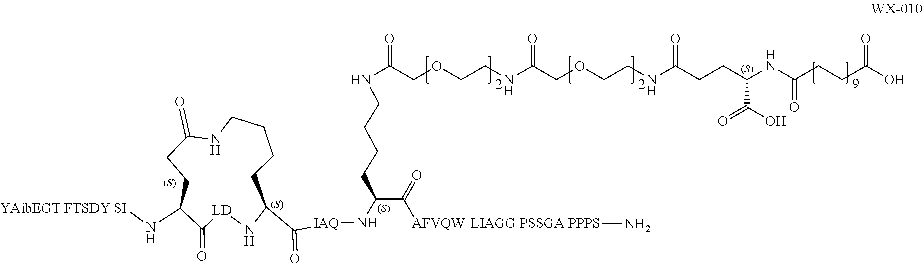

Referring to the synthesis of WX-001, WX-010 was obtained through intermediate B-8. The molecular weight of the polypeptide was confirmed by ESI-MS, calculated for 4839.4, found 4839.6.

Biological Test Data

### Experimental Example 1: GLP-1R/GIPR Agonist Activity Test In Vitro

A. Main Material:

1) Cell Strain

The cell strains were constructed by Shanghai WuXi AppTec. See the table below for details.

| Target | Host Cell | Clone |
|---|---|---|
| GLP-1R | HEK293 | N/A |
| GIPR | CHO | N/A |

2) Reagents and Consumables

| Name | Batch. | Item NO. | Manufacturer |
|---|---|---|---|
| cAMP assay kit | 29F | 62AM4PEJ | Cisbio |
| 1M HEPES | 2120919 | 15630-106 | Invitrogen |
| Hanks balanced salt solution | 2185775 | 14025 | Invitrogen |
| (HBSS) | | | |
| Human serum albumin | SLCF7301 | A1653-10G | Sigma |
| (HSA) | | | |
| Casein | SLCC9458 | C4765-10 mL | Sigma |
| 3-Isobutyl-1-methylxanthine | STBF6061V | I5879-5G | Sigma |
| (IBMX) | | | |
| ECHO qualified 384-well | 0006433672 | PP-0200 | Labcyte |
| plate | | | |
| OptiPlate-384 | 8210-19481 | 6007299 | PerkinElmer |

3) Instruments

| Name | Model | Manufacturer |
|---|---|---|
| EnVision | envision2014 | PerkinElmer |
| Vi-cell counter | Vi-CELL ™ Cell Viability | Beckman |
| | Analyzer | |
| Bravo | Bravo V11 | Agilent |
| ECHO | ECHO 555 | Labcyte |
| Centrifuge | Allegra ™ 25R Centrifuge | Beckman |

B. Method

1) Experimental Materials

Experimental Buffer

| | Storage | | Final |
|---|---|---|---|
| Reagent | Concentration | Volume | Concentration |
| Hanks balanced salt solution | 1× | 48.7 mL/ | About 1× |
| | | 44.7 mL | |
| HEPES buffer | 1 mol/L | 250 μL | 5 mmol/L |
| 5% casein solution (HEPES)/ | 5%/10% | 1000 μL/ | 0.10%/1% |
| 10% human serum albumin | | 5000 μL | |
| solution (HSA) | | | |
| 3-Isobutyl-1-methylxanthine | 500 mmol/L | 50 μL | 0.5 mmol/L |
| (IBMX) | | | |

Detection Reagent Preparation

| | Storage | | Final |
|---|---|---|---|
| Reagent | Concentration | Volume | Concentration |
| Cell lysate | 1× | 9.5 mL | About 1× |
| D2-cAMP solution | 40× | 250 μL | 1× |
| cAMP-antibody solution | 40× | 250 μL | 1× |

Experimental Method

a) Preparation of the Compound Plate:

The test compound was serially diluted 4-fold with 10 points. The initial concentration was 30 μM, and the dilution was completed with Bravo.

b) Compound Transference:

1) 100 nL of the compound was transferred to OptiPlate-384 plate using Echo.

2) The OptiPlate-384 plate was centrifuged at 1000 rpm for 5 seconds.

c) Preparation of Cell Suspension:

1) A GLP-IR/G1PR cell cryopreservation tube was quickly thawed in warm water at 37° C.

2) The cell suspension was transferred to a Transfer 15 mL centrifuge tube and gently rinsed with 10 mL of HBSS.

3) The centrifuge tube was centrifuged at 1000 rpm for 1 minute at room temperature.

4) The supernatant was discarded.

5) The bottom cells were gently dispersed and gently rinsed with 10 mL of HBSS.

The cells were centrifuged to settle, and finally resuspended with the experimental buffer.

Vi-cell was used to measure cell density and activity.

7) The GLP-1R/GIPR cell concentration was diluted to 2.0*10⁵/mL with the experimental buffer.

8) 100 nL of the diluted cell suspension was transferred into the OptiPlate-384 plate.

9) The diluted cell suspension was incubated at room temperature for 30 minutes.

d) Addition of Detection Reagent:

1) 10 μL of 800 nM gradient-diluted cAMP standard was added to the empty well of the OptiPlate-384 plate.

2) 10 μL of cAMP detection reagent was added.

3) The OptiPlate-384 plate was covered with TopSeal-A film and incubated at room temperature for 60 minutes.

TopSeal-A was removed and read in EnVision.

C. Experimental Results

The experimental results are shown in Table 1.

TABLE 1
GLP-1R/GIPR Agonist Activity Test Results in vitro

| | GLP-IR Agonist Activity | | | GIPR Agonist Activity | | |
|---|---|---|---|---|---|---|
| | EC₅₀ (nM) | | | EC₅₀ (nM) | | |
| Polypeptide | (−) 1% | (+) 1% | | (−) 1% | (+) 1% | |
| Drugs | HSA | HSA | Ratio | HSA | HSA | Ratio |
| WX-001 | 0.014 | 3.664 | 265 | 0.44 | 23.04 | 53 |
| WX-002 | 0.052 | NA | NA | 0.29 | NA | NA |
| WX-003 | 0.090 | NA | NA | 0.30 | NA | NA |
| WX-005 | 0.042 | NA | NA | 0.22 | NA | NA |
| WX-006 | 0.051 | NA | NA | 0.45 | NA | NA |
| WX-007 | 0.14 | NA | NA | 0.13 | NA | NA |
| WX-008 | 0.08 | NA | NA | 0.32 | NA | NA |
| WX-010 | 0.03448 | 33.96 | 984.9 | 11.86 | 443 | 37.4 |

Conclusion: The compounds of the present disclosure have strong agonist activity on GLP-1R/GIPR.

### Experimental Example 2: Pharmacokinetic Evaluation of Compounds in Rats

A. Experimental Purpose

To test the pharmacokinetics of compounds in SD rats in vivo

B. Experimental Operation

The pharmacokinetic characteristics of the compounds in rodents after subcutaneous injection were tested according to the standard protocol. In the experiment, the candidate compounds were formulated into clear solutions, and a single subcutaneous injection (SC, 0.048 mpk) was given to the rats. The injection vehicle was citrate buffer (20 mM, pH=7). Whole blood was collected and prepared to obtain plasma. Drug concentration was analyzed by LC-MS/MS method, and pharmacokinetic parameters were calculated by Phoenix WinNonlin software.

C. Experimental Results

The experimental results are shown in Table 2.

TABLE 2
Pharmacokinetic Test Results

| | Maximum Plasma | | SC Concentration |
|---|---|---|---|
| Compound | Concentration | Half-life | Integral |
| Number | (nM) | T_{1/2} (h) | AUC_{0-72 h} (nM · hr) |
| WX001 | 22 | 12 | 728 |
| WX002 | 14 | 17 | 1316 |
| WX005 | 14 | 17 | 1329 |
| WX006 | 16 | 10 | 846 |

Conclusion: The compounds of the present disclosure have excellent pharmacokinetic properties in rats.

### Experimental Example 3: Pharmacokinetic Evaluation of Compounds in Rats

A. Experimental Purpose

To test the pharmacokinetics of compounds in C₅₇BL/6 mice in vivo

B. Experimental Operation

The pharmacokinetic characteristics of the compounds in rodents after intravenous injection and subcutaneous injection were tested according to the standard protocol. In the experiment, the candidate compounds were formulated into clear solutions, and a single intravenous injection (IV, 0.048 mpk) and subcutaneous injection (SC, 0.048 mpk) were given to the mice. The vehicle for intravenous injection was PBS buffer (pH=7), and the vehicle for subcutaneous injection was citrate buffer (20 mM, pH=7). Whole blood was collected and prepared to obtain plasma. Drug concentration was analyzed by LC-MS/MS method, and pharmacokinetic parameters were calculated by Phoenix WinNonlin software.

C. Experimental Results

The experimental results are shown in Table 3.

TABLE 3
Pharmacokinetic Test Results

| | | | SC | |
|---|---|---|---|---|
| | | | Concentration | |
| Compound | Clearance | Half-life | Integral AUC_{0-72 h} | Bioavailability |
| Number | (mL/min/kg) | T_{1/2} (h) | (nM · hr) | F (%) |
| WX001 | 0.088 | 15.5 | 1554 | 85.5% |

Conclusion: The compounds of the present disclosure have excellent pharmacokinetic properties in mice.

### Experimental Example 4: Pharmacokinetic Evaluation of Compounds in Cynomolgus Monkeys

A. Experimental Purpose

To test the pharmacokinetics of compounds in cynomolgus monkeys in vivo

B. Experimental Operation

The pharmacokinetic characteristics of the compounds in mammals after intravenous injection and subcutaneous injection were tested according to the standard protocol. In the experiment, the candidate compounds were formulated into clear solutions, and a single subcutaneous injection (SC, 0.02 mpk) was given to the cynomolgus monkeys. The vehicle for subcutaneous injection was citrate buffer (20 mM, pH=7). Whole blood was collected and prepared to obtain plasma. Drug concentration was analyzed by LC-MS/MS method, and pharmacokinetic parameters were calculated by Phoenix WinNonlin software.

C. Experimental Results

The experimental results are shown in Table 4.

TABLE 4
Pharmacokinetic Test Results

| | | | | SC Concentration |
|---|---|---|---|---|
| Compound | | Tₘₐₓ | Half-life | Integral |
| Number | Cₘₐₓ (nM) | (h) | T_{1/2} (h) | AUC_{0-240 h} (nM · hr) |
| WX001 | 30 | 21 | 80 | 3973 |
| WX002 | 33 | 29 | 81 | 4582 |
| WX005 | 31 | 40 | 98 | 4553 |
| WX006 | 30 | 29 | 72.4 | 3640 |

Conclusion: The compounds of the present disclosure have excellent pharmacokinetic properties in monkeys.

### Experimental Example 5: Plasma Stability (PLS) Test

A. Experimental Purpose

To study the stability of test compounds in the plasma of normal mice.

B. Experimental Operation

1. Before the experiment, the coagulated frozen plasma was thawed in a water bath at 37° C. Plasma was centrifuged at 4000 rpm for 5 min. If there was blood clot, the blood clot was removed and the pH value was adjusted to 7.4±0.1.

2. Preparation of the test compound solution: 100 μM of the solution was prepared by dilution with DMSO.

3. 98 μL of blank control plasma was added to 2 μL of the test compound solution (100 μM), so that the final concentration of the mixed solution of the two reached 2 μM, and the mixed solution was placed in a 37° C. water bath for incubation.

4. At each time point (0, 10, 30, 60, and 120 min), 1(X) μL of H₃PO₄ solution and 800 μL of stop solution (200 ng/mL of tolbutamide and 200 ng/mL of labetalol in 100% methanol solution) were added to precipitate protein, and mixed thoroughly.

5. The samples were centrifuged at 4000 rpm for 20 min, and 1(X) μL of supernatant was taken from each well for LC-MS/MS analysis.

C. Experimental Results

The experimental results are shown in Table 5.

TABLE 5
PLS Test Results

| Compound Number | WX001 | WX002 | WX005 | WX006 |
|---|---|---|---|---|
| PLS (H/M) | >289/>289 | >289/>289 | >289/>289 | >289/>289 |
| T_{1/2} (min) | | | | |

Conclusion: The compounds of the present disclosure have excellent plasma stability.

Experimental Example 6: Plasma Protein Binding (PPB) Test

A. Experimental Purpose

To study the binding of the test compounds to human/mouse plasma albumin.

B. Experimental Operation

1. Matrix preparation: On the day of the experiment, the plasma was thawed in cold water and centrifuged at 3220 rpm for 5 min to remove all blood clots. The pH of the obtained plasma was measured and adjusted to 7.4±0.1 using 1% phosphoric acid or 1N sodium hydroxide as needed.

2. Dilution steps of the test compounds: The test compounds were dissolved in dimethyl sulfoxide (DMSO) to prepare stock solutions at concentrations of 10 mM and 2 mM, respectively. A 40 μM working solution was prepared by diluting 2 μL of the stock solution (2 mM) with 98 μL of DMSO. A 400 μM working solution of the control compound was prepared by diluting 10 μL of the stock solution with 240 μL of DMSO. The working solution of the compound (5 μL) was mixed with the blank matrix (995 μL) at a ratio of 1:200 to prepare the loading matrix.

3. Analysis Steps

3.1 An equal amount of 30 μL of the loading matrix (n=2) was transferred to the sample collection plate to prepare samples of time 0 (TO) for residue determination. The samples were immediately matched to the corresponding blank buffer in a final volume of 60 μL with a 1:1 volume ratio of plasma to buffer in each well. Then, 60 μL of 4% H₃PO₄ in H₂O and 480 μL of stop solution containing an internal standard were then added to the TO samples of the test compounds. They were then stored at 2-8° C. with other samples pending for further processing.

3.2 The remaining plasma samples were pre-incubated in a carbon dioxide incubator at 37±1° C. for 30 min. The prepared protein-free samples (F samples) and loading matrix samples (230 μL) were transferred to polycarbonate tubes (n=2) and ultracentrifuged at 37° C. and 155000×g (35000 rpm) for 4 hours.

3.3 To prepare T samples (test samples), an additional matrix-containing sample was transferred to a separate 96-well plate (sample incubation plate) and incubated at 37° C. for 4 hours.

3.4 After centrifugation, 30 μL of protein-free sample and 30 μL of T sample were transferred from the second layer of supernatant (below the upper layer) to a new sample collection plate. Each sample was mixed with the corresponding blank buffer or matrix in a final volume of 60 μL with a matrix:buffer volume ratio of 1:1. 60 μL of 4% H₃PO₄ aqueous solution and 480 μL of stop solution (containing internal standard) were added to all samples. The mixture was centrifuged at 4000 rpm for 20 min, and 100 μL of the supernatant of each sample was taken for LC-MS/MS analysis.

C. Experimental Results

The experimental results are shown in Table 6. Note: NA means the plasma-protein binding is too high, and no free drug can be detected under a normal concentration of plasma protein.

TABLE 6
PPB Test Results

| Compound Number | WX001 | WX002 | WX005 | WX006 |
|---|---|---|---|---|
| PPB % unbound | NA/NA | NA/0.21% | NA/0.22% | NA/0.35% |
| (H/M) | | | | |

Conclusion: The compounds of the present disclosure have extremely high plasma-protein binding.

### Experimental Example 7: Kidney Homogenate Stability (BHS) Test

A. Experimental Purpose

To study the stability of the test compounds in normal mouse kidney homogenate.

B. Experimental Operation

1. Before the experiment, the frozen kidney homogenate was thawed in a 37° C. water bath.

2. Test compounds: 10 mM stock solution was diluted with DMSO to prepare 1 mM intermediate solution;

3. test compounds: 1 mM intermediate solution was diluted with DMSO to prepare 50 μM dosing solution;

4. at each time point (0, 10, 30, 60, 120, 180, 240 min), 100 μL of 4% H₃PO₄ and 800 μL of stop solution were added to precipitate the protein, and mixed thoroughly.

5. The samples were centrifuged at 4000 rpm for 10 min, and 100 μL of supernatant was taken from each well to the plate. The samples were shaken at 800 rpm for approximately 10 min before submitting for LC-MS/MS test.

C. Experimental Results

The experimental results are shown in Table 7.

TABLE 7
BHS Test Results

| Compound Number | WX001 | WX002 | WX005 | WX006 |
|---|---|---|---|---|
| BHS (T_{1/2} (min)) | 14 | 20 | 21 | 234 |

### Experimental Example 8: Intraperitoneal Glucose Tolerance Test (ipGTT)—in Vivo Pharmacodynamic Evaluation of Compounds in Mice

A. Experimental Purpose

To study the improvement effect of the test compounds on the glucose tolerance of normal mice

B. Experimental Operation

1. After the mice were grouped according to body weight and blood glucose, each group of animals was injected with the test compound (0.3 nmol/kg) and vehicle (20 mM citrate buffer). The mice were fasted overnight, and intraperitoneally injected with glucose solution (2 g/kg, 10 mL/kg) after 18 hours;

2. a glucometer was used to measure blood glucose concentrations at −60, 0, 15, 30, 60 and 120 minutes after glucose administration.

C. Experimental Results

The experimental results are shown in Table 8.

TABLE 8
ipGTT Test Results

| Compound Number | Vehicle | WX001 | WX002 | WX005 | WX006 |
|---|---|---|---|---|---|
| Blood glucose | 1916 | 1277 | 1150 | 1143 | 1164 |
| AUC_{0-120 min} | | | | | |
| (mmol · min) | | | | | |

Conclusion: The compounds of the present disclosure have excellent effects on improving glucose tolerance.

### Experimental Example 9: Pharmacodynamic Study—In Vivo Pharmacodynamic Evaluation in db/db Mice

A. Experimental Purpose

To study the glycemic control effect of the test compounds on type II diabetic db/db mice

B. Experimental Operation

1. After the db/db mice arrived at the facility, they were kept in an animal breeding room with strictly controlled environmental conditions. The temperature in the breeding room was maintained at 20-24° C. and the humidity was maintained at 30-70%. The temperature and humidity in the breeding room were monitored in real-time by a hygrothermograph, and the temperature and humidity were recorded twice a day (one time in the morning and one time in the afternoon). The daylighting in the animal breeding room was controlled by an electronic timing lighting system, and the lights were turned on for 12 hours and turned off for 12 hours every day (turned on at 7:00 in the morning and turned off at 19:00 in the afternoon). During the experiment, the animals were kept in single cages, and toys were provided for each cage. During the experiment, the animals had free access to food (feed for growth/breeding of rats and mice) and drinking water.

2. The vehicle and the test compound (15 nmol/kg) were subcutaneously injected into each group of animals. The administration time: 9:30-11:00 in the morning, and the administration frequency was once a day, and the administration was continuous for 4 weeks.

C. Experimental Results

The experimental results are shown in Table 9.

TABLE 9
Glucose-lowering Effect of the Test Compounds in db/db Mice

| Compound Number | Vehicle | WX001 | WX002 | WX005 | WX006 |
|---|---|---|---|---|---|
| ΔHbA1c % | 15% | −28% | −21% | −21% | −23% |
| (compared with Day 1 | | | | | |
| after four weeks of | | | | | |
| continuous | | | | | |
| administration) | | | | | |

Conclusion: The compounds of the present disclosure exhibit excellent glucose-lowering efficacy in db/db mice.

### Experimental Example 10: Pharmacodynamic Study—In Vivo Pharmacodynamic

Evaluation in DIO Mice

A. Experimental Purpose

To study the weight loss effect of the test compounds in DIO mice

B. Experimental Operation

1. After the DIO mice arrived at the WuXi AppTec facility, they were kept in an animal breeding room with strictly controlled environmental conditions. The temperature in the breeding room was maintained at 20-24° C., and the humidity was maintained at 30-70%. The temperature and humidity in the breeding room were monitored in real-time by a hygrothermograph, and the temperature and humidity were recorded twice a day (one time in the morning and one time in the afternoon). The daylighting in the animal breeding room was controlled by an electronic timing lighting system, and the lights were turned on for 12 hours and turned off for 12 hours every day (turned on at 7:00 in the morning and turned off at 19:00 in the afternoon). During the experiment, the animals were kept in single cages, and toys were provided for each cage. During the experiment, the animals had free access to food (feed for the growth/breeding of rats and mice) and drinking water.

2. The vehicle and the test compound (10 nmol/kg) were subcutaneously injected into each group of animals, respectively. The administration time: 9:30 in the morning, and the administration frequency was once every three days, and the administration cycle was 22 days.

C. Experimental Results

The experimental results are shown in Table 10.

TABLE 10
Pharmacodynamics of the Test Compounds in DIO Mice

| Compound Number | Vehicle | WX001 | WX002 | WX005 | WX006 |
|---|---|---|---|---|---|
| Δ Body weight % | 0.2% | −29% | −26% | −24% | −32% |
| (after 22 days | | | | | |
| compared with day 1) | | | | | |
| Δ Adipose tissue % | 2% | −54% | −48% | −47% | −62% |
| (after 22 days | | | | | |
| compared to day 1) | | | | | |
| Δ Non-adipose tissue | −3% | −11% | −10% | −8% | −18% |
| % (after 22 days | | | | | |
| compared with day 1) | | | | | |

Conclusion: The compounds of the present disclosure exhibit excellent weight loss efficacy in DIO mice.

What is claimed is:

1. A compound represented by the following formula or a pharmaceutically acceptable salt thereof,
SEQ ID NO1: YAibEGT FTSDY SIAibLD KK¹AZ₀K₀ Z₁FZ₂E₀W LZ₃AGG PSSGA PPPS₀
SEQ ID NO2: YAibEGT FTSDY SIAibLD KE₀AQK₀ AFVK¹W LIAGG PSSGA PPPS₀
SEQ ID NO3: YAibEGT FTSDY SIE₀LD KK₀AQK¹ AFVQW LIAGG PSSGA PPPS₀
SEQ ID NO4: YAibEGT FTSDY SIE₀LD K₀IAQK¹ AFVQW LIAGG PSSGA PPPS₀
wherein
Aib has a structure of 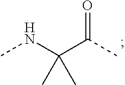
S₀ is selected from 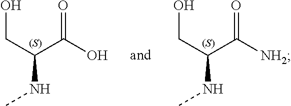
Z₀ is selected from glutamine (Q) and asparagine (N);
Z₁ is selected from alanine (A) and glutamic acid (E);
Z₂ is selected from valine (V) and isoleucine (I);
Z₃ is selected from isoleucine (I) and leucine (L);
E₀ and K₀ indicate that the carboxyl group on the side chain of the glutamic acid and the amino group on the side chain of the lysine jointly form a lactam structure, and in this case, K₀Z₁FZ₂E₀ has a structure of 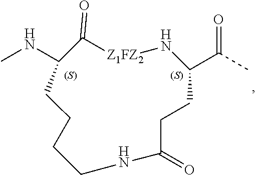
E₀AQK₀ has a structure of 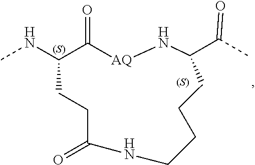
E₀LDKK₀ has a structure of 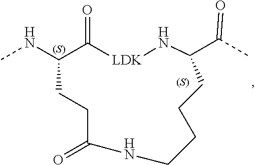
and E₀LDK₀ has a structure of 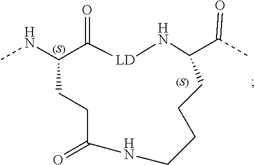
K¹ indicates that the amino group on the side chain of the lysine is connected to —X—X₁—X₂, and has a structure of 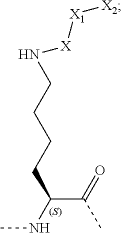
X is selected from 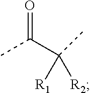
R₁ and R₂ are each independently selected from H and CH₃;
X₁ is selected from 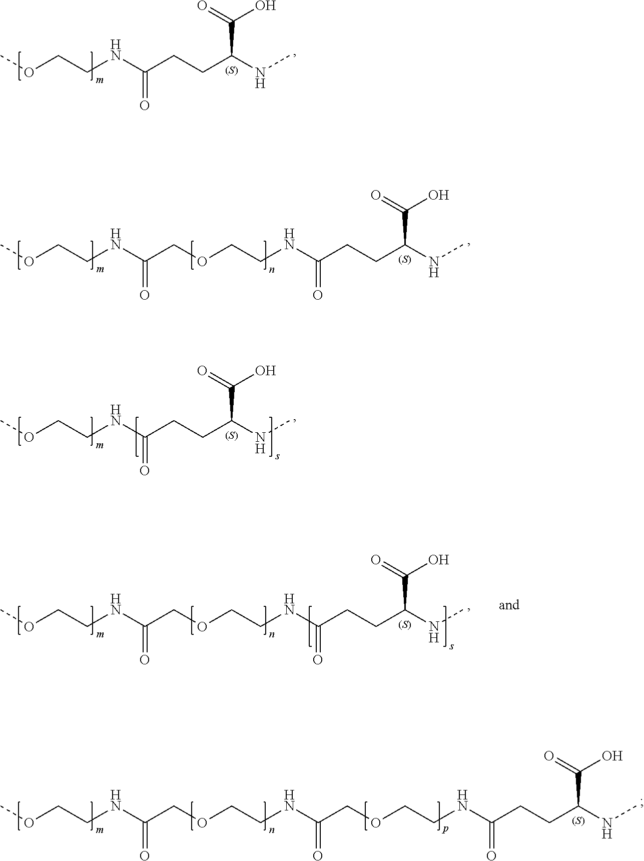
X₂ is selected from 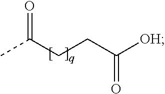
m, n and p are each independently selected from 2 and 3;
s is selected from 2 and 3;
q is selected from 15, 16, 17, 18 and 19.

2. The compound or the pharmaceutically acceptable salt thereof according to claim 1, which is selected from:

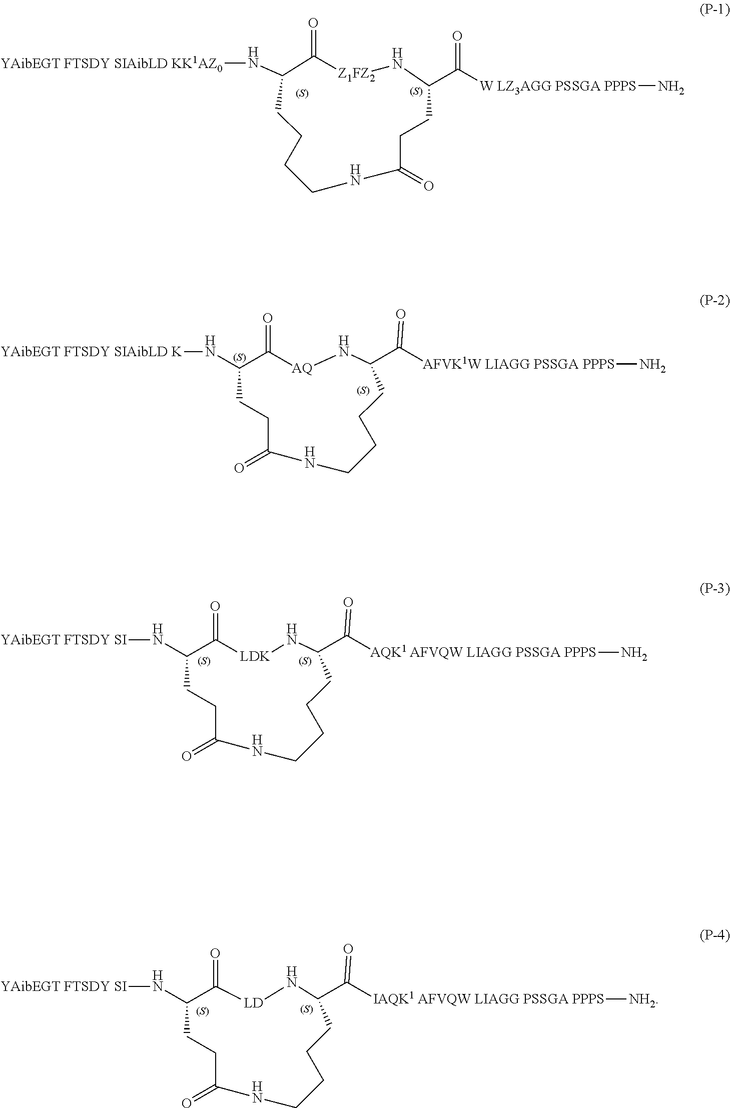

3. The compound or the pharmaceutically acceptable salt thereof according to claim 1, wherein X is selected from —C(═O)—CH₂—.

4. The compound or the pharmaceutically acceptable salt thereof according to claim 1, wherein m, n and p are each independently selected from 2.

5. The compound or the pharmaceutically acceptable salt thereof according to claim 1, wherein s is independently selected from 2.

6. The compound or the pharmaceutically acceptable salt thereof according to claim 1, wherein q is independently selected from 15 and 17.

7. The compound or the pharmaceutically acceptable salt thereof according to claim 1, wherein X₁ is selected from

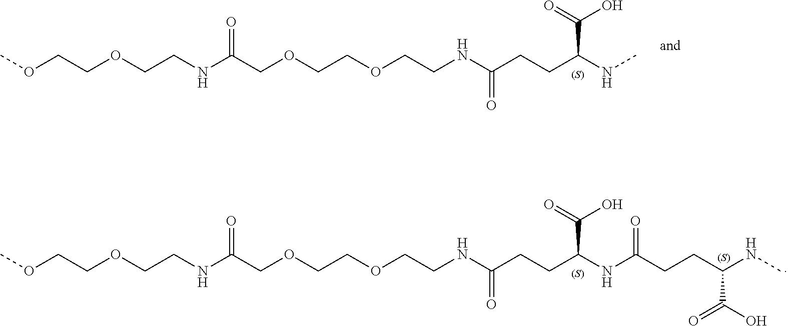

8. The compound or the pharmaceutically acceptable salt thereof according to claim 1, wherein X₂ is selected from

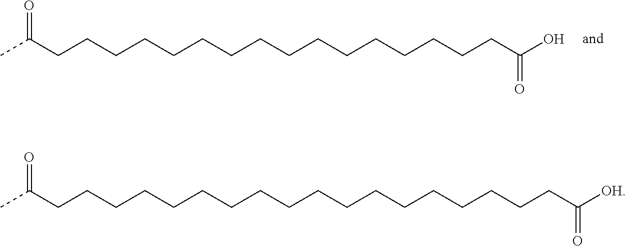

9. The compound or the pharmaceutically acceptable salt thereof according to claim 1, wherein —X—X₁—X₂ is selected from

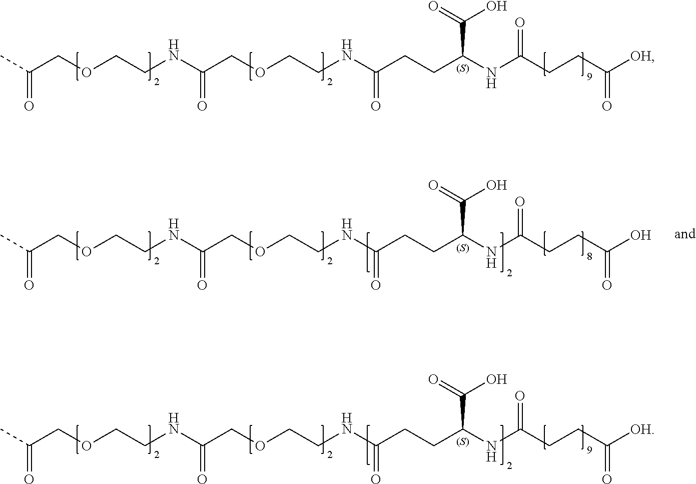

10. A compound represented by the following formula or a pharmaceutically acceptable salt thereof, selected from:

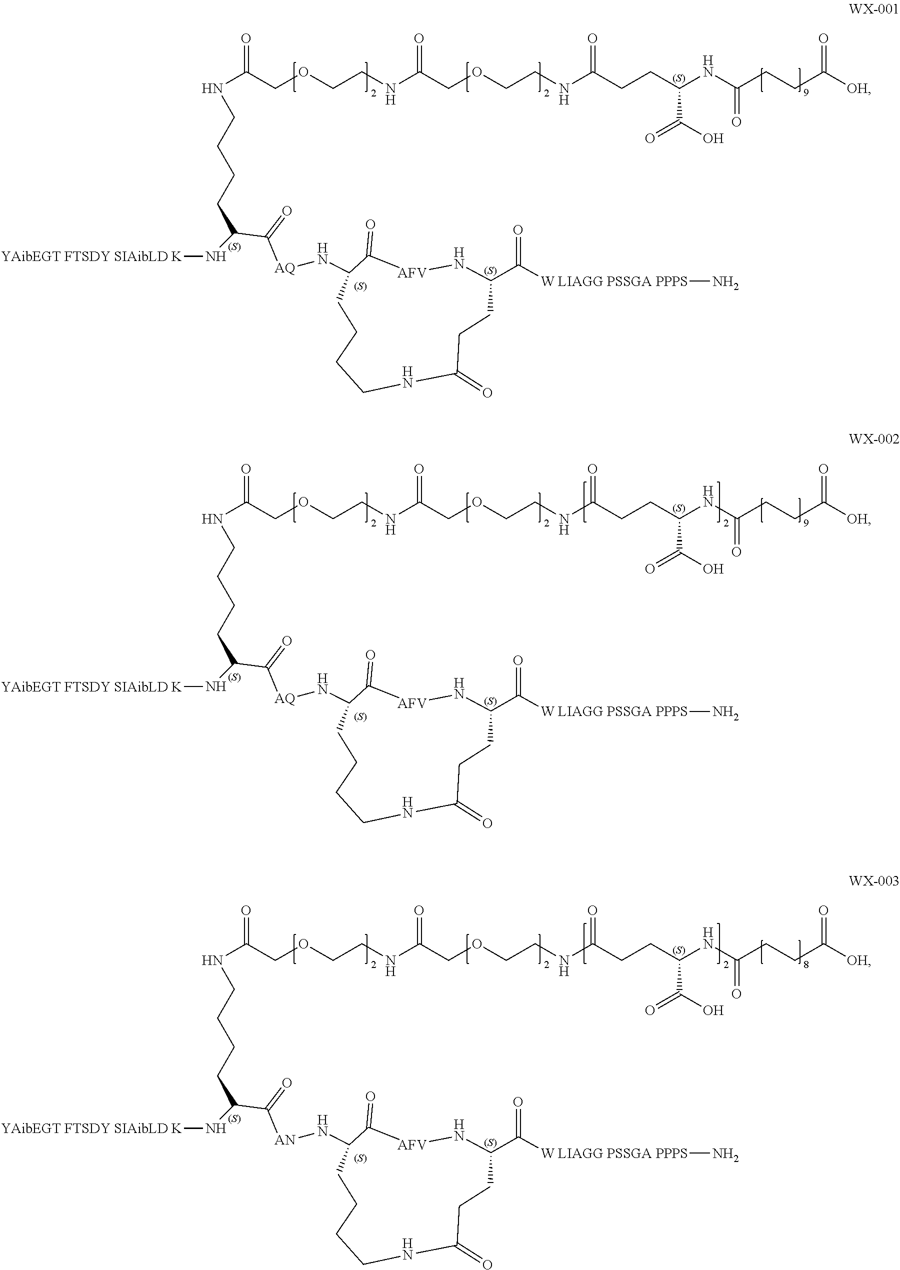

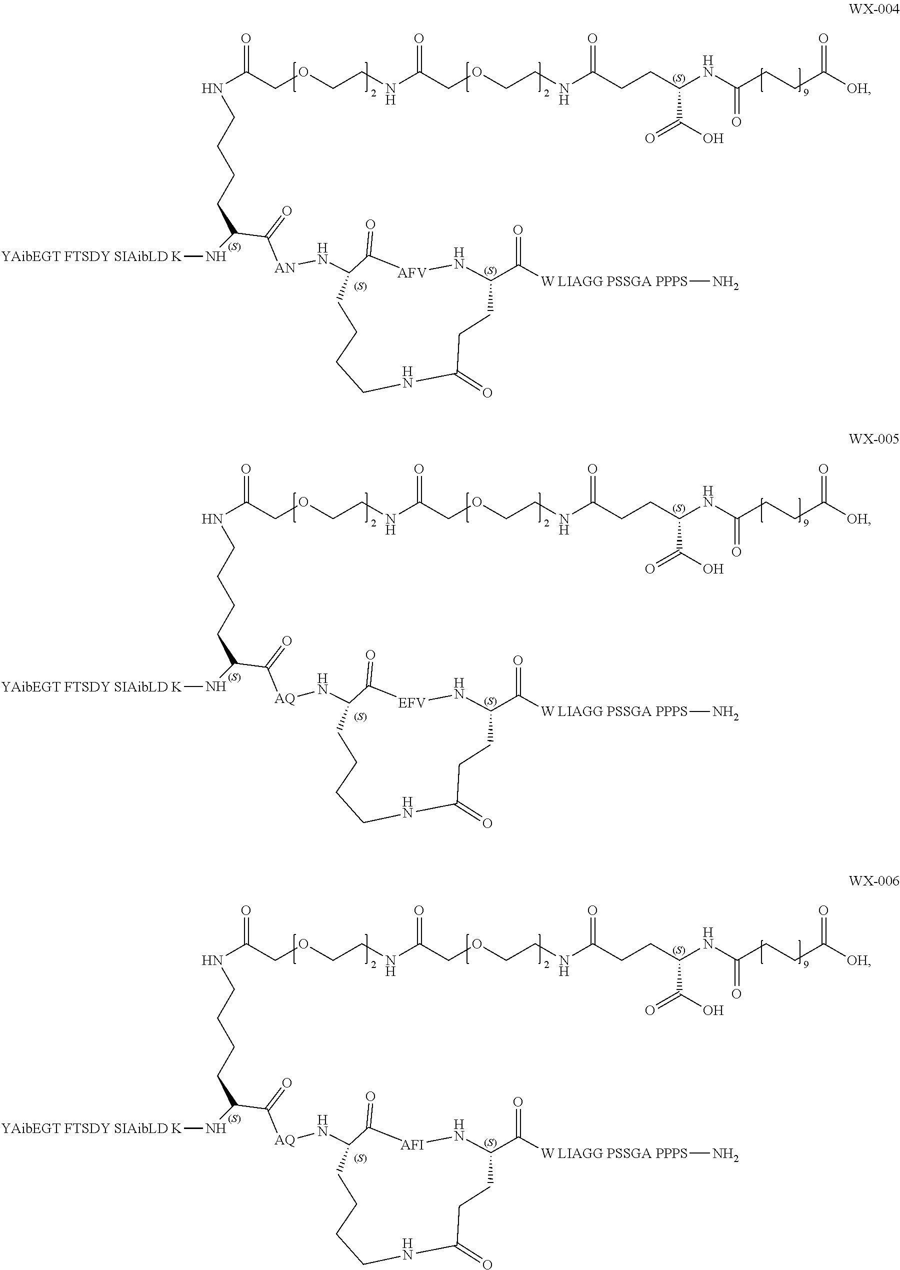

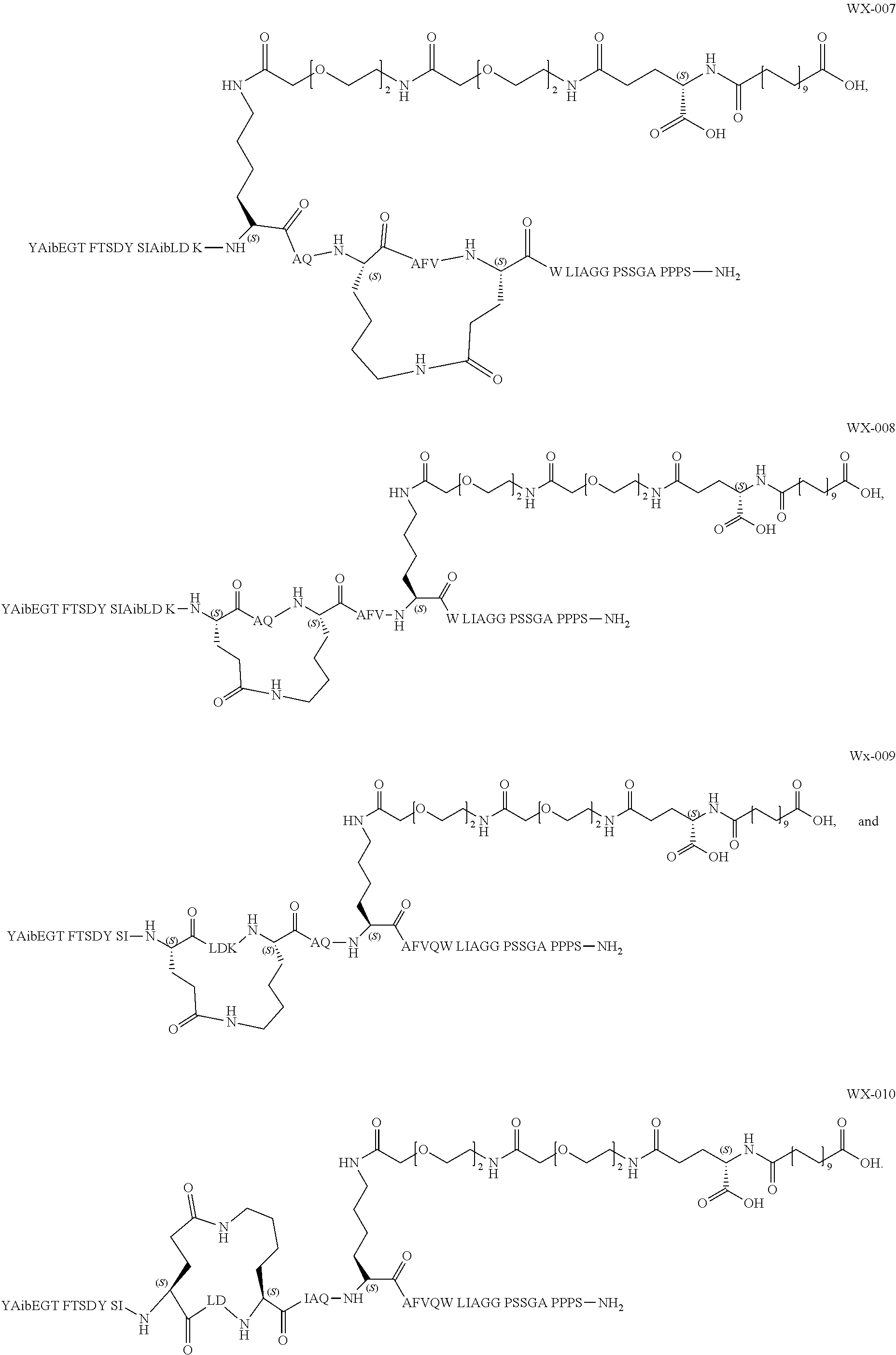

11. A method for treating obesity and diabetes in a subject in need thereof, comprising: administering the compound or the pharmaceutically acceptable salt thereof according to claim 1 to the subject.

12. The compound or the pharmaceutically acceptable salt thereof according to claim 2, wherein X is selected from —C(O)—CH₂—.

13. The compound or the pharmaceutically acceptable salt thereof according to claim 2, wherein m, n and p are each independently selected from 2.

14. The compound or the pharmaceutically acceptable salt thereof according to claim 2, wherein s is independently selected from 2.

15. The compound or the pharmaceutically acceptable salt thereof according to claim 2, wherein q is independently selected from 15 and 17.

16. The compound or the pharmaceutically acceptable salt thereof according to claim 2, wherein X₁ is selected from

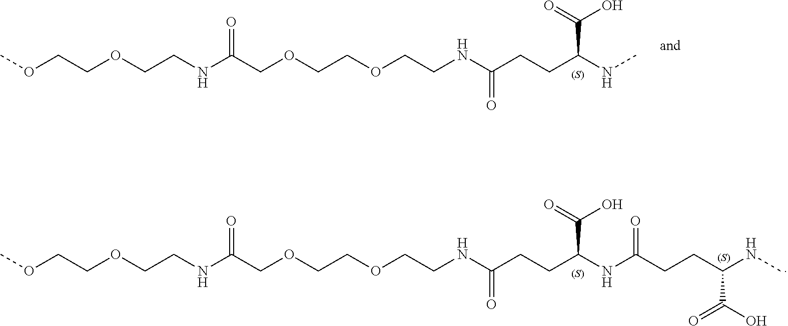

17. The compound or the pharmaceutically acceptable salt thereof according to claim 2, wherein X₂ is selected from

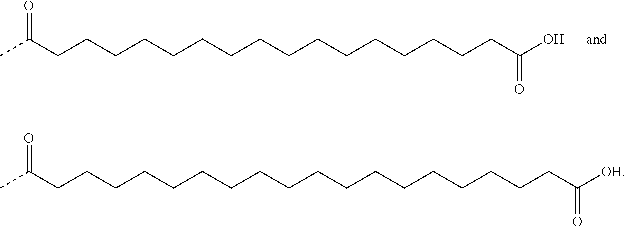

18. The compound or the pharmaceutically acceptable salt thereof according to claim 2, wherein —X—X₁—X₂ is selected from

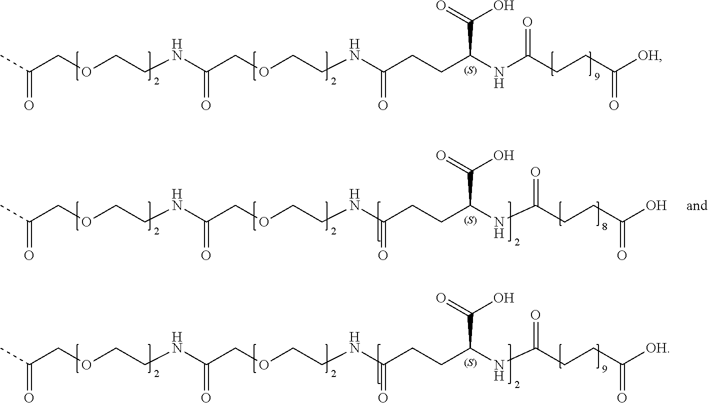

19. A method for treating obesity and diabetes in a subject in need thereof, comprising: administering the compound or the pharmaceutically acceptable salt thereof according to claim 10 to the subject.
